SEQUENCE LISTING (30 sequences)

SEQ ID NO: 1 - protein, 40 aa, artificial sequence
  Amino acid sequence for  HV-GHRH.
HVDAIFTNSYRKVLAQLSARKLLQDILNRQQGERNQEQGA

SEQ ID NO: 2 - protein, 40 aa, artificial sequence
  Amino acid sequence for TI-GHRH.
YIDAIFTNSYRKVLAQLSARKLLQDILNRQQGERNQEQGA

SEQ ID NO: 3 - protein, 40 aa, artificial sequence
  Amino acid sequence for TV-GHRH.
YVDAIFTNSYRKVLAQLSARKLLQDILNRQQGERNQEQGA

SEQ ID NO: 4 - protein, 40 aa, artificial sequence
  Amino acid sequence for 15/27/28-GHRH.
YADAIFTNSYRKVLAQLSARKLLQDILNRQQGERNQEQGA

SEQ ID NO: 5 - protein, 44 aa, artificial sequence
  Consensus sequence for GHRH
TADAIFTNSYRKVLGQLSARKLLQDIMSRQQGESNQERGARARL

SEQ ID NO: 6 - protein, 40 aa, artificial sequence
  Artificial sequence for GHRH (1-40)OH.
XXDAIFTNSYRKVLXQLSARKLLQDIXXRQQGERNQEQGA

SEQ ID NO: 7 - DNA, 323 bp, artificial sequence
  Eukaryotic promoter c5-12.
cggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcg

SEQ ID NO: 8 - DNA, 190 bp, artificial sequence
  Nucleic acid sequence of a hGH poly A tail.
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtaggg

SEQ ID NO: 9 - DNA, 219 bp, artificial sequence
  This is the cDNA for Porcine GHRH.
atggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatga

SEQ ID NO: 10 - protein, 40 aa, artificial sequence
  Amino acid sequence for porcine GHRH.
YADAIFTNSYRKVLGQLSARKLLQDIMSRQQGERNQEQGA

SEQ ID NO: 11 - DNA, 3534 bp, artificial sequence
  Sequence for the HV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 12 - DNA, 3534 bp, artificial sequence
  Sequence for the TI-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatatcgatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 13 - DNA, 3534 bp, artificial sequence
  Nucleic acid sequence for the TV-GHRH plasmid.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgtagatgccatcttcaccaacagctaccggaaggtgctggcccagctgtccgcccgcaagctgctccaggacatcctgaacaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 14 - DNA, 3534 bp, artificial sequence; identical to SEQ ID NO: 12
  Sequence for the 15/27/28 GHRH plasmid.

SEQ ID NO: 15 - DNA, 3534 bp, artificial sequence
  Plasmid sequence for wildtype GHRH.
gttgtaaaacgacggccagtgaattgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgcagatgccatcttcaccaacagctaccggaaggtgctgggccagctgtccgcccgcaagctgctccaggacatcatgagcaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgac

SEQ ID NO: 16 - DNA, 4260 bp, Artificial sequence
  Sequence for the pSP-SEAP cDNA.
ggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatatcaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgta

SEQ ID NO: 17 - DNA, 2710 bp, artificial sequence
  Codon optimized (“GHRH”) sequence for mouse.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctctgggtgctctttgtgatcctcatcctcaccagcggcagccactgcagcctgcctcccagccctcccttcaggatgcagaggcacgtggacgccatcttcaccaccaactacaggaagctgctgagccagctgtacgccaggaaggtgatccaggacatcatgaacaagcagggcgagaggatccaggagcagagggccaggctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 18 - DNA, 2713 bp, artificial sequence
  Codon optimized (“GHRH”) sequence for rat.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttcttcgtgctgctgaccctgaccagcggaagccactgcagcctgcctcccagccctcccttcagggtgcgccggcacgccgacgccatcttcaccagcagctacaggaggatcctgggccagctgtacgctaggaagctcctgcacgagatcatgaacaggcagcagggcgagaggaaccaggagcagaggagcaggttcaactgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 19 - DNA, 2716 bp, artificial sequence
  Codon optimized (“GHRH”) sequence for bovine.
ccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctgtgggtgttcttcctggtgaccctgaccctgagcagcggaagccacggcagcctgcccagccagcccctgaggatccctaggtacgccgacgccatcttcaccaacagctacaggaagatcctgggccagctgagcgctaggaagctcctgcaggacatcatgaacaggcagcagggcgagaggaaccaggagcagggcgcctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccgacaaacaaaccaccgctggtagcggtggtttttttgtttgcaagcagcagattacgcgcagaaaaaaaggatctcaagaagatcctttgatcttttctacggggtctgacgctcagctagcgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcgtgtaatacgactcactatagggcgaattggagct

SEQ ID NO: 20 - DNA, 2716 bp, artificial sequence; identical to SEQ ID NO: 19
  TCodon optimized (“GHRH”) sequence for ovine.

SEQ ID NO: 21 - DNA, 2713 bp, artificial sequence
  Codon optimized (“GHRH”) sequence forchicken.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggccctgtgggtgttctttgtgctgctgaccctgacctccggaagccactgcagcctgccacccagcccacccttccgcgtcaggcgccacgccgacggcatcttcagcaaggcctaccgcaagctcctgggccagctgagcgcacgcaactacctgcacagcctgatggccaagcgcgtgggcagcggactgggagacgaggccgagcccctgagctgataagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggctagcgcttagaagaactcatccagcagacggtagaatgcaatacgttgagagtctggagctgcaataccatacagaaccaggaaacggtcagcccattcaccacccagttcctctgcaatgtcacgggtagccagtgcaatgtcctggtaacggtctgcaacacccagacgaccacagtcaatgaaaccagagaaacgaccattctcaaccatgatgttcggcaggcatgcatcaccatgagtaactaccaggtcctcaccatccggcatacgagctttcagacgtgcaaacagttcagccggtgccagaccctgatgttcctcatccaggtcatcctggtcaaccagacctgcttccatacgggtacgagcacgttcaatacgatgttttgcctggtggtcaaacggacaggtagctgggtccagggtgtgcagacgacgcattgcatcagccatgatagaaactttctctgccggagccaggtgagaagacagcaggtcctgacccggaacttcacccagcagcagccagtcacgaccagcttcagtaactacatccagaactgcagcacacggaacaccagtggttgccagccaagacagacgagctgcttcatcctgcagttcattcagagcaccagacaggtcagttttaacaaacagaactggacgaccctgtgcagacagacggaaaacagctgcatcagagcaaccaatggtctgctgtgcccagtcataaccaaacagacgttcaacccaggctgccggagaacctgcatgcagaccatcctgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

SEQ ID NO: 22 - DNA, 55 bp, artificial sequence
  Sequence for 5′ UTR of hGH.
caaggcccaactccccgaaccactcagggtcctgtggacagctcacctagctgcc

SEQ ID NO: 23 - DNA, 782 bp, artificial sequence
  Nucleic acid sequence of a plasmid pUC-18 origin of replicaition
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccttt

SEQ ID NO: 24 - DNA, 5 bp, artificial sequence
  This is a NEO ribosomal binding site
tcctc

SEQ ID NO: 25 - DNA, 29 bp, artificial sequence
  Nucleic acid sequence of a prokaryotic PNEO promoter.
accttaccagagggcgccccagctggcaa

SEQ ID NO: 26 - DNA, 3558 bp, artificial sequence
  Sequence for the inducible pGR1774 with human GHRH
atgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccatgtagaactagtgatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgcagatgccatcttcaccaacagctaccggaaggtgctgggccagctgtccgcccgcaagctgctccaggacatcatgagcaggcagcagggagagagcaaccaagagcgaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattcgataccgtcgacctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatycgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattaattcgagcttgcatgcctgcagggtcgaagcggagtactgtcctccgagtggagtactgtcctccgagcggagtactgtcctccgagtcgagggtcgaagcggagtactgtcctccgagtggagtactgt

SEQ ID NO: 27 - DNA, 4855 bp, artificial Sequence
  Sequence for the muscle-specific GeneSwitch - pGS1633
aggggccgctctagctagagtctgcctgccccctgcctggcacagcccgtacctggccgcacgctccctcacaggtgaagctcgaaaactccgtccccgtaaggagccccgctgccccccgaggcctcctccctcacgcctcgctgcgctcccggctcccgcacggccctgggagaggcccccaccgcttcgtccttaacgggcccggcggtgccgggggattatttcggccccggccccgggggggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggtgctcccgcccgcctcgataaaaggctccggggccggcgggcgactcagatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagctaccggtccaccatggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggatcctccggactagaaaagccgaattctgcaggaattgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgcgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgaattcgagcttgcatgcctgc

SEQ ID NO: 28 - DNA, 2739 bp, artificial sequence
  Codon optimized plasmid for porcine GHRH.
ccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggtatgcagatgccatcttcaccaacagctaccggaaggtgctgggccagctgtccgcccgcaagctgctccaggacatcatgagcaggcagcagggagagaggaaccaagagcaaggagcataatgactgcaggaattcgatatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccgacaaacaaaccaccgctggtagcggtggtttttttgtttgcaagcagcagattacgcgcagaaaaaaaggatctcaagaagatcctttgatcttttctacggggtctgacgctcagctagcgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcgtgtaatacgactcactatagggcgaattggagct

SEQ ID NO: 29 - DNA, 3534 bp, artificial sequence; identical to SEQ ID NO: 11
  Codon optimized plasmid for GHRH expression.

SEQ ID NO: 30 - DNA, 2725 bp, artificial sequence
  Codon optimized plasmid for GHRH.
tgtaatacgactcactatagggcgaattggagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggatcccaaggcccaactccccgaaccactcagggtcctgtggacagctcacctagctgccatggtgctctgggtgttcttctttgtgatcctcaccctcagcaacagctcccactgctccccacctccccctttgaccctcaggatgcggcggcacgtagatgccatcttcaccaacagctaccggaaggtgctggcccagctgtccgcccgcaagctgctccaggacatcctgaacaggcagcagggagagaggaaccaagagcaaggagcataatgacatcaagcttatcggggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccggtaccagcttttgttccctttagtgagggttaatttcgagcttggtcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccgacaaacaaaccaccgctggtagcggtggtttttttgtttgcaagcagcagattacgcgcagaaaaaaaggatctcaagaagatcctttgatcttttctacggggtctgacgctcagctagcgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagcaactgttgggaagggcgatcg

(19) United States
(12) Patent Application Publication
(10) Pub. No.: US 2004/0204358 A1
(43) Pub. Date: Oct. 14, 2004

(54) Reducing culling in herd animals growth hormone releasing hormone (GHRH)

(75) Inventors: Patricia A. Brown, Conroe, TX (US); Ruxandra Draghia-Akli, Houston, TX (US); Robert H. Carpenter, Bastrop, TX (US)

Correspondence Address: T. Ling Chwang, Jackson Walker L.L.P., #600, 2435 North Central Expressway, Richardson, TX, 75080, US

(73) Assignee: ADViSYS, Inc., The Woodlands, TX

(21) Appl. No.: 10/764,818

(22) Filed: Jan. 26, 2004

Related U.S. Application Data
(60) Provisional application No. 60/443,104, filed on Jan. 28, 2003.

(51) Int. Cl. A61K 38/00

(52) U.S. Cl. 514/12

(57) ABSTRACT

One aspect of the current invention is a method of decreasing an involuntary cull rate in farm animals, wherein the involuntary cull results from infection, disease, morbidity, or mortality. Additionally, milk production, animal welfare, and body condition scores are improved by utilizing methodology that administers the isolated nucleic acid expression construct encoding a GHRH or functional biological equivalent to an animal through a parenteral route of administration. Following a single dose of nucleic acid expression vector, animals are healthier and effects are demonstrated long term without additional administration(s) of the expression construct.

### BACKGROUND

Dairy Cow Culling: A decision to voluntarily cull selected animals from a herd is rarely based upon any single criteria. Although not wanting to be bound by theory, the biological and market factors surrounding a voluntary culling decision are both complex and unpredictable. Additionally, the dynamic nature of such factors include uncertainty regarding future productivity and economic value for the herd. For example, by determining a production level where a particular dairy cow is not profitable would be a key determination step for having the animal left in the milking string, dried off or sold. There are many reasons for culling animals, and some of these reasons are loosely separated into “involuntary culling” and “voluntary culling” categories. Examples of “involuntary” culling include: being crippled (poor feet and legs); persistent mastitis problems; non-breeders; and disease or death. Examples of “voluntary” culling include selling animals for breeding stock or selling lower producing animals to make room for a higher producing replacement animal. Other general examples for culling are summarized in Table 1. Although not wanting to be bound by theory, several general models have been developed that list multiple voluntary culling categories, which can be used to help the dairymen make voluntary culling decisions. Generally, when an animal falls into more than one of the above culling categories, the animal is typically a good candidate for sale or slaughter at the packing plant. If strict culling criteria are used on a consistent basis, unprofitable animals can be removed from the dairy herd in timely fashion, and may still retain some economic or “salvage” value due to sale or slaughter at the packing plant. In contrast, an involuntary cull due to disease or death typically results in no economic or salvage value. Additionally, diseased animals may reduced the welfare of the entire herd.

Involuntary culling is a major economic problem in dairy industry. Although the average overall cull rate in North America is approximately 36% (Radke and Shook, 2001), most culling is involuntary in nature. Due to the high percentage of involuntary culling, voluntary cull decisions that revolving around rational economic parameters (e.g. maintenance of herd size) are typically held to a minimum. When a plasmid mediated growth hormone releasing hormone (“GHRH”) treatment is given to dairy cows, the treated animals show a reduced number of involuntary culls in a herd, wherein the culls were due to disease/injury or death. The GHRH treatment can be of extraordinary economical importance to the dairyman (FIG. 10) and gainfully contribute to the general welfare of the herd.

TABLE 1

| | Average % of | |
|---|---|---|
| REASON | Total culls | Average % culls |
| VOLUNTARY | | |
| Dairy Sales | 13.7 | 4.9 |
| Low production | 25.4 | 9.1 |
| Total voluntary | 39.1 | 14.1 |
| INVOLUNTARY | | |
| Reproduction | 22.9 | 8.2 |
| Mastitis/udder | 15.0 | 5.4 |
| Disease/injury | 10.4 | 3.7 |
| Death | 3.3 | 1.2 |
| Feet and legs | 1.8 | 0.7 |
| Temperament | 0.2 | 0.1 |
| Miscellaneous | 7.3 | 2.6 |
| Total involuntary | 60.9 | 21.9 |

Growth Hormone Releasing Hormone (“GHRH”) and Growth Hormone (“GH”) Axis: To better understand utilizing GHRH plasmid mediated gene supplementation as a treatment to decrease involuntary culling, the mechanisms and current understanding of the GHRH/GH axis will be addressed. Although not wanting to be bound by theory, the central role of growth hormone (“GH”) is controlling somatic growth in humans and other vertebrates. The physiologically relevant pathways regulating GH secretion from the pituitary are fairly well known. The GH production pathway is composed of a series of interdependent genes whose products are required for normal growth. The GH pathway genes include: (1) ligands, such as GH and insulin-like growth factor-I (“IGF-I”); (2) transcription factors such as prophet of pit 1, or prop 1, and pit 1: (3) agonists and antagonists, such as growth hormone releasing hormone (“GHRH”) and somatostatin (“S S”), respectively; and (4) receptors, such as GHRH receptor (“GHRH-R”) and the GH receptor (“GH-R”). These genes are expressed in different organs and tissues, including the hypothalamus, pituitary, liver, and bone. Effective and regulated expression of the GH pathway is essential for optimal linear growth, as well as homeostasis of carbohydrate, protein, and fat metabolism. GH synthesis and secretion from the anterior pituitary is stimulated by GHRH and inhibited by somatostatin, both hypothalamic hormones. GH increases production of IGF-I, primarily in the liver, and other target organs. IGF-I and GH, in turn, feedback on the hypothalamus and pituitary to inhibit GHRH and GH release. GH elicits both direct and indirect actions on peripheral tissues, the indirect effects being mediated mainly by IGF-I.

The immune function is modulated by IGF-I, which has two major effects on B cell development: potentiation and maturation, and as a B-cell proliferation cofactor that works together with interlukin-7 (“IL-7”). These activities were identified through the use of anti-IGF-I antibodies, antisense sequences to IGF-I, and the use of recombinant IGF-I to substitute for the activity. There is evidence that macrophages are a rich source of IGF-I. The treatment of mice with recombinant IGF-I confirmed these observations as it increased the number of pre-B and mature B cells in bone marrow (Jardieu et al., 1994). The mature B cell remained sensitive to IGF-I as immunoglobulin production was also stimulated by IGF-I in vitro and in vivo (Robbins et al., 1994).

The production of recombinant proteins in the last 2 decades provided a useful tool for the treatment of many diverse conditions. For example, GH-deficiencies in short stature children, anabolic agent in burn, sepsis, and AIDS patients. However, resistance to GH action has been reported in malnutrition and infection. GH replacement therapy is widely used clinically, with beneficial effects, but therapy is associated with several disadvantages: GH must be administered subcutaneously or intramuscularly once a day to three times a week for months, or usually years; insulin resistance and impaired glucose tolerance; accelerated bone epiphysis growth and closure in pediatric patients (Blethen and MacGillivray, 1997; Blethen and Rundle, 1996).

In contrast, essentially no side effects have been reported for recombinant GHRH therapies. Extracranially secreted GHRH, as mature peptide or truncated molecules (as seen with pancreatic islet cell tumors and variously located carcinoids) are often biologically active and can even produce acromegaly (Esch et al., 1982; Thorner et al., 1984). Administration of recombinant GHRH to GH-deficient children or adult humans augments IGF-I levels, increases GH secretion proportionally to the GHRH dose, yet still invokes a response to bolus doses of recombinant GHRH (Bercu and Walker, 1997). Thus, GHRH administration represents a more physiological alternative of increasing subnormal GH and IGF-I levels (Corpas et al., 1993).

GH is released in a distinctive pulsatile pattern that has profound importance for its biological activity (Argente et al., 1996). Secretion of GH is stimulated by the GHRH, and inhibited by somatostatin, and both hypothalamic hormones (Thorner et al., 1995). GH pulses are a result of GHRH secretion that is associated with a diminution or withdrawal of somatostatin secretion. In addition, the pulse generator mechanism is timed by GH-negative feedback. Effective and regulated expression of the GH and insulin-like growth factor-I (“IGF-I”) pathway is essential for optimal linear growth, homeostasis of carbohydrate, protein, and fat metabolism, and for providing a positive nitrogen balance (Murray and Shalet, 2000). Numerous studies in humans, sheep or pigs showed that continuous infusion with recombinant GHRH protein restores the normal GH pattern without desensitizing GHRH receptors or depleting GH supplies as this system is capable of feed-back regulation, which is abolished in the GH therapies (Dubreuil et al., 1990; Vance, 1990; Vance et al., 1985). Although recombinant GHRH protein therapy entrains and stimulates normal cyclical GH secretion with virtually no side effects, the short half-life of GHRH in vivo requires frequent (one to three times a day) intravenous, subcutaneous or intranasal (requiring 300-fold higher dose) administration. Thus, as a chronic treatment, GHRH administration is not practical.

Wild type GHRH has a relatively short half-life in the circulatory system, both in humans (Frohman et al., 1984) and in farm animals. After 60 minutes of incubation in plasma 95% of the GHRH(1-44)NH2 is degraded, while incubation of the shorter (1-40)OH form of the hormone, under similar conditions, shows only a 77% degradation of the peptide after 60 minutes of incubation (Frohman et al., 1989). Incorporation of cDNA coding for a particular protease-resistant GHRH analog in a therapeutic nucleic acid vector results in a molecule with a longer half-life in serum, increased potency, and provides greater GH release in plasmid-injected animals (Draghia-Akli et al., 1999), herein incorporated by reference. Mutagenesis via amino acid replacement of protease sensitive amino acids prolongs the serum half-life of the GHRH molecule. Furthermore, the enhancement of biological activity of GHRH is achieved by using super-active analogs that may increase its binding affinity to specific receptors (Draghia-Akli et al., 1999).

Direct plasmid DNA gene transfer is currently the basis of many emerging nucleic acid therapy strategies and thus does not require viral genes or lipid particles (Aihara and Miyazaki, 1998; Muramatsu et al., 2001). Skeletal muscle is target tissue, because muscle fiber has a long life span and can be transduced by circular DNA plasmids that express over months or years in an immunocompetent host (Davis et al., 1993; Tripathy et al., 1996). Previous reports demonstrated that human GHRH cDNA could be delivered to muscle by an injectable myogenic expression vector in mice where it transiently stimulated GH secretion to a modes extent over a period of two weeks (Draghia-Akli et al., 1997).

Administering novel GHRH analog proteins (U.S. Pat. Nos. 5,847,066; 5846,936; 5,792,747; 5,776,901; 5,696,089; 5,486,505; 5,137,872; 5,084,442, 5,036,045; 5,023,322; 4,839,344; 4,410,512, RE33,699) or synthetic or naturally occurring peptide fragments of GHRH (U.S. Pat. Nos. 4,833,166; 4,228,158; 4,228,156; 4,226,857; 4,224,316; 4,223,021; 4,223,020; 4,223, 019) for the purpose of increasing release of growth hormone have been reported. A GHRH analog containing the following mutations have been reported (U.S. Pat. No. 5,846,936): Tyr at position 1 to His; Ala at position 2 to Val, Leu, or others; Asn at position 8 to Gln, Ser, or Thr; Gly at position 15 to Ala or Leu; Met at position 27 to Nle or Leu; and Ser at position 28 to Asn. The GHRH analog is the subject of U.S. patent application Ser. No. 09/624,268 (“the '268 patent application”), which teaches application of a GHRH analog containing mutations that improve the ability to elicit the release of growth hormone. In addition, the '268 patent application relates to the treatment of growth deficiencies; the improvement of growth performance; the stimulation of production of growth hormone in an animal at a greater level than that associated with normal growth; and the enhancement of growth utilizing the administration of growth hormone releasing hormone analog and is herein incorporated by reference.

U.S. Pat. No. 5,061,690 is directed toward increasing both birth weight and milk production by supplying to pregnant female mammals an effective amount of human GHRH or one of it analogs for 10-20 days. Application of the analogs lasts only throughout the lactation period. However, multiple administrations are presented, and there is no disclosure regarding administration of the growth hormone releasing hormone (or factor) as a DNA molecule, such as with plasmid mediated therapeutic techniques.

U.S. Pat. No. 5,134,120 (“the '120 patent”) and U.S. Pat. No. 5,292,721 (“the '721 patent”) teach that by deliberately increasing growth hormone in swine during the last 2 weeks of pregnancy through a 3 week lactation resulted in the newborn piglets having marked enhancement of the ability to maintain plasma concentrations of glucose and free fatty acids when fasted after birth. In addition, the 120 and 721 patents teach that treatment of the sow during lactation results in increased milk fat in the colostrum and an increased milk yield. These effects are important in enhancing survivability of newborn pigs and weight gain prior to weaning. However the 120 and 721 patents provide no teachings regarding administration of the growth hormone releasing hormone as a DNA form.

Growth Hormone (“GH”) and Growth Hormone Releasing Hormone (“GHRH”) in Farm animals: The administration of recombinant growth hormone (“GH”) or recombinant GH has been used in farm animals for many years, but not as a pathway to decrease involuntary culling, or to increase the herd welfare. More specifically, recombinant GH treatment in farm animals has been shown to enhance lean tissue deposition and/or milk production, while increasing feed efficiency (Etherton et al., 1986; Klindt et al., 1998). Numerous studies have shown that recombinant GH markedly reduces the amount of carcass fat; and consequently the quality of products increases. However, chronic GH administration has practical, economical and physiological limitations that potentially mitigate its usefulness and effectiveness (Chung et al., 1985; Gopinath and Etherton, 1989b). Experimentally, recombinant GH-releasing hormone (“GHRH”) has been used as a more physiological alternative. The use of GHRH in large animal species (e.g. pigs or cattle) not only enhances growth performance and milk production, but more importantly, the efficiency of production from both a practical and metabolic perspective (Dubreuil et al., 1990; Farmer et al., 1992). For example, the use of recombinant GHRH in lactating sows has beneficial effects on growth of the weanling pigs, yet optimal nutritional and hormonal conditions are needed for GHRH to exert its full potential (Farmer et al., 1996).

Comparisons of recombinant GH and GHRH treatments have been conducted in cattle. For example, one group of Holstein cows received 12 mg/d of GHRH as continuous i.v. infusion for 60 days, and another group of Holstein cows received 14 mg/d of bovine GH as a single daily i.m. injection for 60 days. The different GH and GHRH treatments resulted in similar milk composition, body condition score, and body weight. However, cows that received the i.v. infusion of 12 mg/d of GHRH had greater galactopoietic activity than cows receiving i.m. injections of 14 mg/d of bovine GH (Dahl et al., 1991). This observation was also made in beef cattle, wherein GH response to 4.5 microg/100 kg body weight challenge dose of GHRH was positively related to sire milk daily rate (Auchtung et al., 2001). Consequently, the high cost of the recombinant peptides and the required frequency of administration currently limit the widespread use of this treatment. The introduction of bovine somatotropin (bovine GH, bST) in production animals has raised concerns over increased levels of hormones (i.e. GH and IGF-I) in the meat or milk produced by treated animals. Although levels of insulin-like growth factor I (IGF-I) in meat and milk were marginally increased by bST treatment, research has shown the IGF-I is not orally active when fed to rats, even at doses ranging from 200 to 2,000 microgram/kg for 14 days (Hammond et al., 1990). Nevertheless, the sudden increase in GH and IGF-I levels after recombinant protein administration is concerning. These major drawbacks can be obviated by using a gene delivery and in vivo expression approach to direct the chronic ectopic production of GHRH.

Gene Delivery and in vivo Expression: Recently, the delivery of specific genes to somatic tissue in a manner that can correct inborn or acquired deficiencies and imbalances was proved to be possible (Herzog et al., 2001; Song et al., 2001; Vilquin et al., 2001). Gene-based drug delivery offers a number of advantages over the administration of recombinant proteins. These advantages include the conservation of native protein structure, improved biological activity, avoidance of systemic toxicities, and avoidance of infectious and toxic impurities. In addition, nucleic acid vector therapy allows for prolonged exposure to the protein in the therapeutic range, because the newly secreted protein is present continuously in the blood circulation. In a few cases, the relatively low expression levels achieved after simple plasmid injection, are sufficient to reach physiologically acceptable levels of bioactivity of secreted peptides, especially for vaccine purposes (Danko and Wolff, 1994; Tsurumi et al., 1996).

The primary limitation of using recombinant protein is the limited availability of protein after each administration. Nucleic acid vector therapy using injectable DNA plasmid vectors overcomes this, because a single injection into the patient's skeletal muscle permits physiologic expression for extensive periods of time (WO 99/05300 and WO 01/06988). Injection of the vectors promotes the production of enzymes and hormones in animals in a manner that more closely mimics the natural process. Furthermore, among the non-viral techniques for gene transfer in vivo, the direct injection of plasmid DNA into muscle tissue is simple, inexpensive, and safe.

In a plasmid-based expression system, a non-viral gene vector may be composed of a synthetic gene delivery system in addition to the nucleic acid encoding a therapeutic gene product. In this way, the risks associated with the use of most viral vectors can be avoided. The non-viral expression vector products generally have low toxicity due to the use of “species-specific” components for gene delivery, which minimizes the risks of immunogenicity generally associated with viral vectors. Additionally, no integration of plasmid sequences into host chromosomes has been reported in vivo to date, so that this type of nucleic acid vector therapy should neither activate oncogenes nor inactivate tumor suppressor genes. As episomal systems residing outside the chromosomes, plasmids have defined pharmacokinetics and elimination profiles, leading to a finite duration of gene expression in target tissues.

Efforts have been made to enhance the delivery of plasmid DNA to cells by physical means including electroporation, sonoporation, and pressure. Administration by electroporation involves the application of a pulsed electric field to create transient pores in the cellular membrane without causing permanent damage to the cell. It thereby allows for the introduction of exogenous molecules (Smith and Nordstrom, 2000). By adjusting the electrical pulse generated by an electroporetic system, nucleic acid molecules can travel through passageways or pores in the cell that are created during the procedure. U.S. Pat. No. 5,704,908 describes an electroporation apparatus for delivering molecules to cells at a selected location within a cavity in the body of a patient. These pulse voltage injection devices are also described in U.S. Pat. Nos. 5,439,440 and 5,702,304, and PCT WO 96/12520, 96/12006, 95/19805, and 97/07826.

Recently, significant progress has been obtained using electroporation to enhance plasmid delivery in vivo. Electroporation has been used very successfully to transfect tumor cells after injection of plasmid (Lucas et al., 2002; Matsubara et al., 2001)) or to deliver the anti-tumor drug bleomycin to cutaneous and subcutaneous tumors in humans (Gehl et al., 1998; Heller et al., 1996). Electroporation also has been extensively used in mice (Lesbordes et al., 2002; Lucas et al., 2001; Vilquin et al., 2001), rats (Terada et al., 2001; Yasui et al., 2001), and dogs (Fewell et al., 2001) to deliver therapeutic genes that encode for a variety of hormones, cytokines or enzymes. Our previous studies using growth hormone releasing hormone (GHRH) showed that plasmid therapy with electroporation is scalable and represents a promising approach to induce production and regulated secretion of proteins in large animals and humans (Draghia-Akli et al., 1999; Draghia-Akli et al., 2002b).

The ability of electroporation to enhance plasmid uptake into the skeletal muscle has been well documented, as described above. In addition, plasmid formulated with poly-L-glutamate (“PLG”) or polyvinylpyrolidone (“PVP”) has been observed to increase plasmid transfection and consequently expression of the desired transgene. The anionic polymer sodium PLG could enhance plasmid uptake at low plasmid concentrations, while reducing any possible tissue damage caused by the procedure. PLG is a stable compound and resistant to relatively high temperatures (Dolnik et al., 1993). PLG has been previously used to increase stability in vaccine preparations (Matsuo et al., 1994) without increasing their immunogenicity. It also has been used as an anti-toxin post-antigen inhalation or exposure to ozone (Fryer and Jacoby, 1993). In addition, plasmid formulated with PLG or polyvinylpyrrolidone (“PVP”) has been observed to increase gene transfection and consequently gene expression to up to 10 fold in the skeletal muscle of mice, rats and dogs (Fewell et al., 2001; Mumper et al., 1998). PLG has been used to increase stability of anti-cancer drugs (Li et al., 2000) and as “glue” to close wounds or to prevent bleeding from tissues during wound and tissue repair (Otani et al., 1996; Otani et al., 1998).

Although not wanting to be bound by theory, PLG will increase the transfection of the plasmid during the electroporation process, not only by stabilizing the plasmid DNA, and facilitating the intracellular transport through the membrane pores, but also through an active mechanism. For example, positively charged surface proteins on the cells could complex the negatively charged PLG linked to plasmid DNA through protein-protein interactions. When an electric field is applied, the surface proteins reverse direction and actively internalize the DNA molecules, process that substantially increases the transfection efficiency. Furthermore, PLG will prevent the muscle damage associated with in vivo plasmid delivery (Draghia-Akli et al., 2002a) and will increase plasmid stability in vitro prior to injection.

The use of directly injectable DNA plasmid vectors has been limited in the past. The inefficient DNA uptake into muscle fibers after simple direct injection has led to relatively low expression levels (Prentice et al., 1994; Wells et al., 1997) In addition, the duration of the transgene expression has been short (Wolff et al., 1990). The most successful previous clinical applications have been confined to vaccines (Danko and Wolff, 1994; Tsurumi et al., 1996).

Although there are references in the art directed to electroporation of eukaryotic cells with linear DNA (McNally et al., 1988; Neumann et al., 1982) (Toneguzzo et al., 1988) (Aratani et al., 1992; Nairn et al., 1993; Xie and Tsong, 1993; Yorifuji and Mikawa, 1990), these examples illustrate transfection into cell suspensions, cell cultures, and the like, and the transfected cells are not present in a somatic tissue.

U.S. Pat. No. 4,956,288 is directed to methods for preparing recombinant host cells containing high copy number of a foreign DNA by electroporating a population of cells in the presence of the foreign DNA, culturing the cells, and killing the cells having a low copy number of the foreign DNA.

U.S. Pat. No. 5,874,534 (“the '534 patent”) and U.S. Pat. No. 5,935,934 (“the '934 patent”) describe mutated steroid receptors, methods for their use and a molecular switch for nucleic acid vector therapy, the entire content of each is hereby incorporated by reference. A molecular switch for regulating expression in nucleic acid vector therapy and methods of employing the molecular switch in humans, animals, transgenic animals and plants (e.g. GeneSwitch®) are described in the '534 patent and the '934 patent. The molecular switch is described as a method for regulating expression of a heterologous nucleic acid cassette for nucleic acid vector therapy and is comprised of a modified steroid receptor that includes a natural steroid receptor DNA binding domain attached to a modified ligand binding domain. The modified binding domain usually binds only non-natural ligands, anti-hormones or non-native ligands. One skilled in the art readily recognizes natural ligands do not readily bind the modified ligand-binding domain and consequently have very little, if any, influence on the regulation or expression of the gene contained in the nucleic acid cassette.

In summary, decrease culling rates, increased body scores, increased milk production, and the improvement of welfare in a herd animal were previously uneconomical and restricted in scope. The related art has shown that it is possible to improve these different conditions in a limited capacity utilizing recombinant protein technology, but these treatments have some significant drawbacks. It has also been taught that nucleic acid expression constructs that encode recombinant proteins are viable solutions to the problems of frequent injections and high cost of traditional recombinant therapy. The introduction of point mutations into the encoded recombinant proteins was a significant step forward in producing proteins that are more stable in vivo than the wild type counterparts. Unfortunately, each amino acid alteration in a given recombinant protein must be evaluated individually, because the related art does not teach one skilled in the art to accurately predict how changes in structure (e.g. amino-acid sequences) will lead to changed functions (e.g. increased or decreased stability) of a recombinant protein. Therefore, the beneficial effects of nucleic acid expression constructs that encode expressed proteins can only be ascertained through direct experimentation. There is a need in the art to expanded treatments for subjects with a disease by utilizing nucleic acid expression constructs that are delivered into a subject and express stable therapeutic proteins in vivo.

### SUMMARY

One aspect of the current invention is a method of decreasing an involuntary cull rate in farm animals, wherein the involuntary cull results from infection, disease, morbidity, or mortality. The method generally comprises delivering into a tissue of the farm animals an isolated nucleic acid expression construct that encodes a growth-hormone-releasing-hormone (“GHRH”) or functional biological equivalent thereof. Specific embodiments of this invention encompass various modes of delivering into the tissue of the farm animals the isolated nucleic acid expression construct (e.g. an electroporation method, a viral vector, in conjunction with a carrier, by parenteral route, or a combination thereof). In a first preferred embodiment, the isolated nucleic acid expression construct is delivered via an electroporation method comprising: a) penetrating the tissue in the farm animal with a plurality of needle electrodes, wherein the plurality of needle electrodes are arranged in a spaced relationship; b) introducing the isolated nucleic acid expression construct into the tissue between the plurality of needle electrodes; and c) applying an electrical pulse to the plurality of needle electrodes. A second preferred embodiments includes the isolated nucleic acid expression construct being delivered in a single dose, and the single dose comprising a total of about a 2 mg of nucleic acid expression construct. Generally the isolated nucleic acid expression construct is delivered into a tissue of the farm animals comprising diploid cells (e.g. muscle cells). In a third specific embodiment the isolated nucleic acid expression construct used for transfection comprises a HV-GHRH plasmid (SEQ ID#11). Other specific embodiments utilize other nucleic acid expression constructs (e.g. an optimized pAV0204 bGHRH plasmid (SEQ ID#19); a TI-GHRH plasmid (SEQ ID#12); TV-GHRH Plasmid (SEQ ID#13); 15/27/28 GHRH plasmid (SEQ ID#14); pSP-wt-GHRH plasmid; an optimized pAV0202 mGHRH plasmid (SEQ ID#17), pAV0203 RGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28). In a fourth specific embodiment, the isolated nucleic acid expression construct further comprises, a transfection-facilitating polypeptide (e.g. a charged polypeptide, or poly-L-glutamate). After delivering the isolated nucleic acid expression construct into the tissues of the farm animals, expression of the encoded GHRH or functional biological equivalent thereof is initiated. The encoded GHRH comprises a biologically active polypeptide; and the encoded functional biological equivalent of GHRH is a polypeptide that has been engineered to contain a distinct amino acid sequence while simultaneously having similar or improved biologically activity when compared to the GHRH polypeptide. One embodiment of a specific encoded GHRH or functional biological equivalent thereof is of formula (SEQ ID No: 6). The farm animal comprises a food animal, or a work animal (e.g. a pig, cow, sheep, goat or chicken).

A second aspect of the current invention includes a method of improving a body condition score (“BCS”) in farm animals comprising: delivering into a tissue of the farm animals an isolated nucleic acid expression construct that encodes a growth-hormone-releasing-hormone (“GHRH”) or functional biological equivalent thereof; wherein the BSC is an aid used to evaluate an overall nutritional state of the farm animal. The method generally comprises delivering into a tissue of the farm animals an isolated nucleic acid expression construct that encodes a growth-hormone-releasing-hormone (“GHRH”) or functional biological equivalent thereof. Specific embodiments of the second aspect of this invention encompass various modes of delivering into the tissue of the farm animals the isolated nucleic acid expression construct (e.g. an electroporation method, a viral vector, in conjunction with a carrier, by parenteral route, or a combination thereof). In a fifth preferred embodiment, the isolated nucleic acid expression construct is delivered via an electroporation method comprising: a) penetrating the tissue in the farm animal with a plurality of needle electrodes, wherein the plurality of needle electrodes are arranged in a spaced relationship; b) introducing the isolated nucleic acid expression construct into the tissue between the plurality of needle electrodes; and c) applying an electrical pulse to the plurality of needle electrodes. A sixth preferred embodiments includes the isolated nucleic acid expression construct being delivered in a single dose, and the single dose comprising a total of about a 2 mg of nucleic acid expression construct. Generally the isolated nucleic acid expression construct is delivered into a tissue of the farm animals comprising diploid cells (e.g. muscle cells). In a seventh specific embodiment the isolated nucleic acid expression construct used for transfection comprises a HV-GHRH plasmid (SEQ ID#11). Other specific embodiments utilize other nucleic acid expression constructs (e.g. an optimized pAV0204 bGHRH plasmid (SEQ ID#19); a TI-GHRH plasmid (SEQ ID#12); TV-GHRH Plasmid (SEQ ID#13); 15/27/28 GHRH plasmid (SEQ ID#14); pSP-wt-GHRH plasmid; an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28). In a eighth specific embodiment, the isolated nucleic acid expression construct further comprises, a transfection-facilitating polypeptide (e.g. a charged polypeptide, or poly-L-glutamate). After delivering the isolated nucleic acid expression construct into the tissues of the farm animals, expression of the encoded GHRH or functional biological equivalent thereof is initiated. The encoded GHRH comprises a biologically active polypeptide; and the encoded functional biological equivalent of GHRH is a polypeptide that has been engineered to contain a distinct amino acid sequence while simultaneously having similar or improved biologically activity when compared to the GHRH polypeptide. One embodiment of a specific encoded GHRH or functional biological equivalent thereof is of formula (SEQ ID No: 6). The farm animal comprises a food animal, or a work animal (e.g. a pig, cow, sheep, goat or chicken).

A third aspect of the current invention includes a method of increasing milk production in a dairy cow comprising: delivering into muscle tissues of the dairy cow an isolated nucleic acid expression construct that encodes a growth-hormone-releasing-hormone (“GHRH”) or functional biological equivalent thereof. The method generally comprises delivering into a tissue of the dairy cow an isolated nucleic acid expression construct that encodes a growth-hormone-releasing-hormone (“GHRH”) or functional biological equivalent thereof. Specific embodiments of the third aspect of this invention encompass various modes of delivering into the tissue of the farm animals the isolated nucleic acid expression construct (e.g. an electroporation method, a viral vector, in conjunction with a carrier, by parenteral route, or a combination thereof). In a ninth preferred embodiment, the isolated nucleic acid expression construct is delivered via an electroporation method comprising: a) penetrating the tissue in the farm animal with a plurality of needle electrodes, wherein the plurality of needle electrodes are arranged in a spaced relationship; b) introducing the isolated nucleic acid expression construct into the tissue between the plurality of needle electrodes; and c) applying an electrical pulse to the plurality of needle electrodes. A tenth preferred embodiments includes the isolated nucleic acid expression construct being delivered in a single dose, and the single dose comprising a total of about a 2 mg of nucleic acid expression construct. Generally the isolated nucleic acid expression construct is delivered into a muscle tissue of the dairy cow comprising diploid cells (e.g. muscle cells). In a eleventh specific embodiment the isolated nucleic acid expression construct used for transfection comprises a HV-GHRH plasmid (SEQ ID#11). Other specific embodiments utilize other nucleic acid expression constructs (e.g. an optimized pAV0204 bGHRH plasmid (SEQ ID#19); a TI-GHRH plasmid (SEQ ID#12); TV-GHRH Plasmid (SEQ ID#13); 15/27/28 GHRH plasmid (SEQ ID#14); pSP-wt-GHRH plasmid; an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28). In a twelfth specific embodiment, the isolated nucleic acid expression construct further comprises, a transfection-facilitating polypeptide (e.g. a charged polypeptide, or poly-L-glutamate). After delivering the isolated nucleic acid expression construct into the tissues of the farm animals, expression of the encoded GHRH or functional biological equivalent thereof is initiated. The encoded GHRH comprises a biologically active polypeptide; and the encoded functional biological equivalent of GHRH is a polypeptide that has been engineered to contain a distinct amino acid sequence while simultaneously having similar or improved biologically activity when compared to the GHRH polypeptide. One embodiment of a specific encoded GHRH or functional biological equivalent thereof is of formula (SEQ ID No: 6).

### BRIEF DESCRIPTION OF THE DRAWINGS

FIG. 1 shows the mortality percentage of heifers, calves at birth, and calves post-natal;

FIG. 2 shows the body condition scores (“BCS”) in heifers treated with pSP-HV-GHRH versus controls at 60-80 days in milk (“DIM”);

FIG. 3 shows the percentage of cows with foot problems during the course of the study;

FIG. 4 shows the overall hoof score improvement in treated animals and controls;

FIG. 5 shows the total involuntary culling rates in heifers treated with pSP-HV-GHRH versus controls at 120 days in milk;

FIG. 6 shows the milk production in animals treated with pSP-HV-GHRH versus controls at different time points (30-120 DIM);

FIG. 7 show the percentage of increased milk production in treated cows versus controls at 30-120 DIM;

FIG. 8 shows the average daily gains in calves born to treated heifers versus those born to control heifers;

FIG. 9 shows an economic model indicating the additional milk production resulting from previously depicted benefits;

FIG. 10 shows an economic model indicating savings in dollars based on a reduced number of involuntary culls;

FIG. 11 shows milk production in pounds of milk produced per day in the individual pairs of treated and control cows paired for parity and calving date;

FIG. 12 shows milk production in treated and control cows paired for parity and calving date;

FIG. 13 shows the average milk IGF-I levels from cows treated with pGHRH and bST;

FIG. 14 shows the maximum milk IGF-I levels from cows treated with pGHRH and bST;

FIG. 15 shows the mean CD2 cell count in control and treated cows;

FIG. 16 shows the mean CD25⁺/CD4₊ cells in control and treated cows;

FIG. 17 shows the mean R⁻/4⁺ in groups control and treated cows;

FIG. 18 shows the mean R+/CD4+ cells in control and treated cows.

### DETAILED DESCRIPTION OF THE PREFERRED EMBODIMENTS

It will be readily apparent to one skilled in the art that various substitutions and modifications may be made in the invention disclosed herein without departing from the scope and spirit of the invention.

The term “a” or “an” as used herein in the specification may mean one or more. As used herein in the claim(s), when used in conjunction with the word “comprising”, the words “a” or “an” may mean one or more than one. As used herein “another” may mean at least a second or more.

The term “analog” as used herein includes any mutant of GHRH, or synthetic or naturally occurring peptide fragments of GHRH, such as HV-GHRH (SEQ ID#1), TI-GHRH (SEQ ID#2), TV-GHRH (SEQ ID#3), 15/27/28-GHRH (SEQ ID#4), (1-44)NH2 (SEQ ID#5) or (1-40)OH (SEQ ID#6) forms, or any shorter form to no less than (1-29) amino acids.

The term “bodily fat proportion” as used herein is defined as the body fat mass divided by the total body weight.

The term “body condition score” (BCS) as used herein is defined as a method to evaluate the overall nutrition and management of dairy heifers and cows.

The term “cassette” as used herein is defined as one or more transgene expression vectors.

The term “cell-transfecting pulse” as used herein is defined as a transmission of a force which results in transfection of a vector, such as a linear DNA fragment, into a cell. In some embodiments, the force is from electricity, as in electroporation, or the force is from vascular pressure.

The term “coding region” as used herein refers to any portion of the DNA sequence that is transcribed into messenger RNA (mRNA) and then translated into a sequence of amino acids characteristic of a specific polypeptide.

The term “cull” as used herein is defined as the removal of an animal from the herd because of sale, slaughter, or death.

The term “delivery” or “delivering” as used herein is defined as a means of introducing a material into a tissue, a subject, a cell or any recipient, by means of chemical or biological process, injection, mixing, electroporation, sonoporation, or combination thereof, either under or without pressure.

The term “DNA fragment” or “nucleic acid expression construct” as used herein refers to a substantially double stranded DNA molecule. Although the fragment may be generated by any standard molecular biology means known in the art, in some embodiments the DNA fragment or expression construct is generated by restriction digestion of a parent DNA molecule. The terms “expression vector,” “expression cassette,” or “expression plasmid” can also be used interchangeably. Although the parent molecule may be any standard molecular biology DNA reagent, in some embodiments the parent DNA molecule is a plasmid. The term “chronically ill” as used herein is defined as patients with conditions as chronic obstructive pulmonary disease, chronic heart failure, stroke, dementia, rehabilitation after hip fracture, chronic renal failure, rheumatoid arthritis, and multiple disorders in the elderly, with doctor visits and/or hospitalization once a month for at least two years.

The term “donor-subject” as used herein refers to any species of the animal kingdom wherein cells have been removed and maintained in a viable state for any period of time outside the subject.

The term “donor-cells” as used herein refers to any cells that have been removed and maintained in a viable state for any period of time outside the donor-subject.

The term “electroporation” as used herein refers to a method that utilized electric pulses to deliver a nucleic acid sequence into cells.

The terms “electrical pulse” and “electroporation” as used herein refer to the administration of an electrical current to a tissue or cell for the purpose of taking up a nucleic acid molecule into a cell. A skilled artisan recognizes that these terms are associated with the terms “pulsed electric field” “pulsed current device” and “pulse voltage device.” A skilled artisan recognizes that the amount and duration of the electrical pulse is dependent on the tissue, size, and overall health of the recipient subject, and furthermore knows how to determine such parameters empirically.

The term “encoded GHRH” as used herein is a biologically active polypeptide of growth hormone releasing hormone.

The term “functional biological equivalent” of GHRH as used herein is a polypeptide that has a distinct amino acid sequence from a wild type GHRH polypeptide while simultaneously having similar or improved biological activity when compared to the GHRH polypeptide. The functional biological equivalent may be naturally occurring or it may be modified by an individual. A skilled artisan recognizes that the similar or improved biological activity as used herein refers to facilitating and/or releasing growth hormone or other pituitary hormones. A skilled artisan recognizes that in some embodiments the encoded functional biological equivalent of GHRH is a polypeptide that has been engineered to contain a distinct amino acid sequence while simultaneously having similar or improved biological activity when compared to the GHRH polypeptide. Methods known in the art to engineer such a sequence include site-directed mutagenesis.

The term “growth hormone” (“GH”) as used herein is defined as a hormone that relates to growth and acts as a chemical messenger to exert its action on a target cell.

The term “growth hormone releasing hormone” (“GHRH”) as used herein is defined as a hormone that facilitates or stimulates release of growth hormone, and in a lesser extent other pituitary hormones, as prolactin.

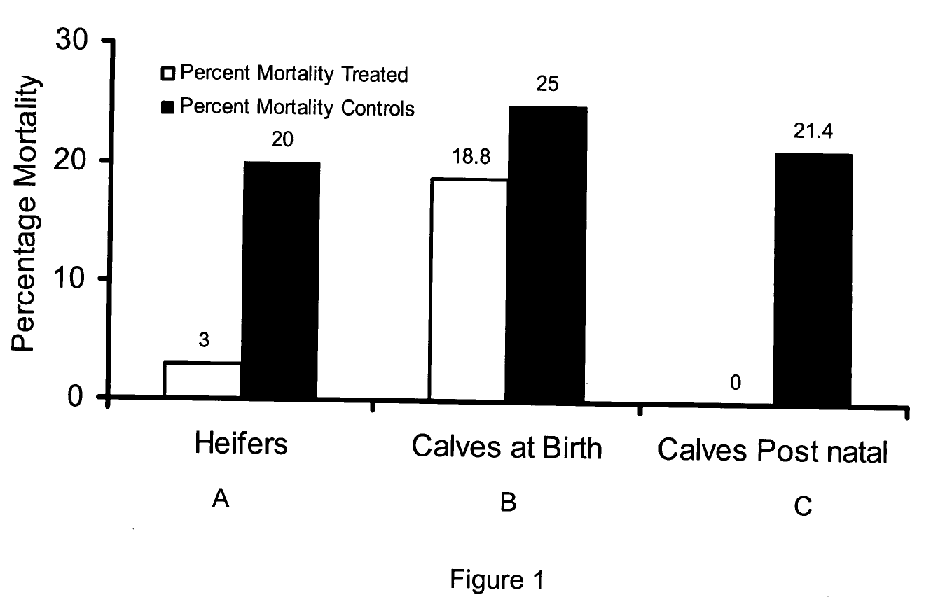

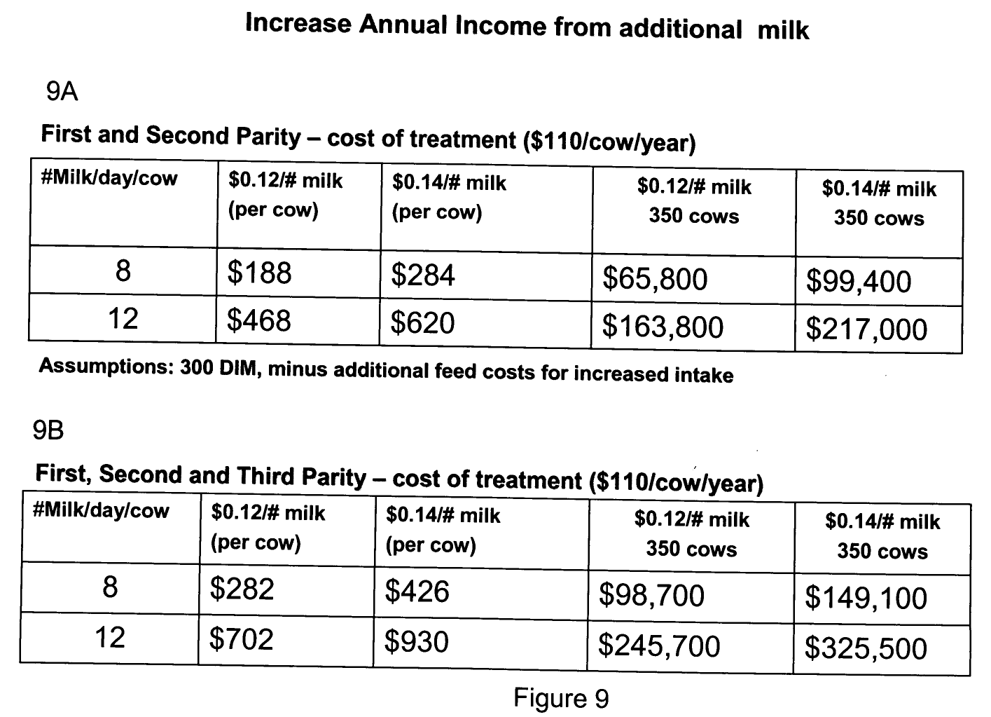

The term “GeneSwitch®” (a registered trademark of Valentis, Inc.; Burlingame, Calif.) as used herein refers to the technology of a mifepristone-inducible heterologous nucleic acid sequences encoding regulator proteins, GHRH, biological equivalent or combination thereof. Such a technology is schematically diagramed in FIG. 1 and FIG. 9. A skilled artisan recognizes that antiprogesterone agent alternatives to mifepristone are available, including onapristone, ZK112993, ZK98734, and 5α pregnane-3,2-dione.

The term “growth hormone” (“GH”) as used herein is defined as a hormone that relates to growth and acts as a chemical messenger to exert its action on a target cell. In a specific embodiment, the growth hormone is released by the action of growth hormone releasing hormone.

The term “growth hormone releasing hormone” (“GHRH”) as used herein is defined as a hormone that facilitates or stimulates release of growth hormone, and in a lesser extent other pituitary hormones, such as prolactin.

The term “heterologous nucleic acid sequence” as used herein is defined as a DNA sequence comprising differing regulatory and expression elements.

The term “immunotherapy” as used herein refers to any treatment that promotes or enhances the body's immune system to build protective antibodies that will reduce the symptoms of a medical condition and/or lessen the need for medications.

The term “involuntary culling” as used herein refers at the removal of a heifer or cow from the study because of disease, injury or death.

The term “lean body mass” (“LBM”) as used herein is defined as the mass of the body of an animal attributed to non-fat tissue such as muscle.

The term “modified cells” as used herein is defined as the cells from a subject that have an additional nucleic acid sequence introduced into the cell.

The term “modified-donor-cells” as used herein refers to any donor-cells that have had a GHRH-encoding nucleic acid sequence delivered.

The term “molecular switch” as used herein refers to a molecule that is delivered into a subject that can regulate transcription of a gene.

The term “nucleic acid expression construct” as used herein refers to any type of genetic construct comprising a nucleic acid coding for a RNA capable of being transcribed. The term “expression vector” can also be used interchangeably herein. In specific embodiments, the isolated nucleic acid expression construct comprises: a promoter; a nucleotide sequence of interest; and a 3′ untranslated region; wherein the promoter, the nucleotide sequence of interest, and the 3′ untranslated region are operatively linked; and in vivo expression of the nucleotide sequence of interest is regulated by the promoter.

The term “operatively linked” as used herein refers to elements or structures in a nucleic acid sequence that are linked by operative ability and not physical location. The elements or structures are capable of, or characterized by accomplishing a desired operation. It is recognized by one of ordinary skill in the art that it is not necessary for elements or structures in a nucleic acid sequence to be in a tandem or adjacent order to be operatively linked.

The term “poly-L-glutamate (“PLG”)” as used herein refers to a biodegradable polymer of L-glutamic acid that is suitable for use as a vector or adjuvant for DNA transfer into cells with or without electroporation.

The term “post-injection” as used herein refers to a time period following the introduction of a nucleic acid cassette that contains heterologous nucleic acid sequence encoding GHRH or a biological equivalent thereof into the cells of the subject and allowing expression of the encoded gene to occur while the modified cells are within the living organism.

The term “plasmid” as used herein refers generally to a construction comprised of extra-chromosomal genetic material, usually of a circular duplex of DNA that can replicate independently of chromosomal DNA. Plasmids, or fragments thereof, may be used as vectors. Plasmids are double-stranded DNA molecule that occur or are derived from bacteria and (rarely) other microorganisms. However, mitochondrial and chloroplast DNA, yeast killer and other cases are commonly excluded.

The term “plasmid mediated gene supplementation” as used herein refers a method to allow a subject to have prolonged exposure to a therapeutic range of a therapeutic protein by utilizing an isolated nucleic acid expression construct in vivo.

The term “pulse voltage device,” or “pulse voltage injection device” as used herein relates to an apparatus that is capable of causing or causes uptake of nucleic acid molecules into the cells of an organism by emitting a localized pulse of electricity to the cells. The cell membrane then destabilizes, forming passageways or pores. Conventional devices of this type are calibrated to allow one to select or adjust the desired voltage amplitude and the duration of the pulsed voltage. The primary importance of a pulse voltage device is the capability of the device to facilitate delivery of compositions of the invention, particularly linear DNA fragments, into the cells of the organism.

The term “plasmid backbone” as used herein refers to a sequence of DNA that typically contains a bacterial origin of replication, and a bacterial antibiotic selection gene, which are necessary for the specific growth of only the bacteria that are transformed with the proper plasmid. However, there are plasmids, called mini-circles, that lack both the antibiotic resistance gene and the origin of replication (Darquet et al., 1997; Darquet et al., 1999; Soubrier et al., 1999). The use of in vitro amplified expression plasmid DNA (i.e. non-viral expression systems) avoids the risks associated with viral vectors. The non-viral expression systems products generally have low toxicity due to the use of “species-specific” components for gene delivery, which minimizes the risks of immunogenicity generally associated with viral vectors. One aspect of the current invention is that the plasmid backbone does not contain viral nucleotide sequences.

The term “promoter” as used herein refers to a sequence of DNA that directs the transcription of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

The term “replication element” as used herein comprises nucleic acid sequences that will lead to replication of a plasmid in a specified host. One skilled in the art of molecular biology will recognize that the replication element may include, but is not limited to a selectable marker gene promoter, a ribosomal binding site, a selectable marker gene sequence, and a origin of replication.

The term “residual linear plasmid backbone” as used herein comprises any fragment of the plasmid backbone that is left at the end of the process making the nucleic acid expression plasmid linear.

The term “recipient-subject” as used herein refers to any species of the animal kingdom wherein modified-donor-cells can be introduced from a donor-subject.

The term “regulator protein” as used herein refers to any protein that can be used to control the expression of a gene.

The term “regulator protein” as used herein refers to protein that increasing the rate of transcription in response to an inducing agent.

The term “secretagogue” as used herein refers to an agent that stimulates secretion. For example, a growth hormone secretagogue is any molecule that stimulates the release of growth hormone from the pituitary when delivered into an animal. Growth hormone releasing hormone is a growth hormone secretagogue.

The terms “subject” or “animal” as used herein refers to any species of the animal kingdom. In preferred embodiments, it refers more specifically to humans and domesticated animals used for: pets (e.g. cats, dogs, etc.); work (e.g. horses, etc.); food (cows, chicken, fish, lambs, pigs, etc); and all others known in the art.

The term “tissue” as used herein refers to a collection of similar cells and the intercellular substances surrounding them. A skilled artisan recognizes that a tissue is an aggregation of similarly specialized cells for the performance of a particular function. For the scope of the present invention, the term tissue does not refer to a cell line, a suspension of cells, or a culture of cells. In a specific embodiment, the tissue is electroporated in vivo. In another embodiment, the tissue is not a plant tissue. A skilled artisan recognizes that there are four basic tissues in the body: 1) epithelium; 2) connective tissues, including blood, bone, and cartilage; 3) muscle tissue; and 4) nerve tissue. In a specific embodiment, the methods and compositions are directed to transfer of linear DNA into a muscle tissue by electroporation.

The term “therapeutic element” as used herein comprises nucleic acid sequences that will lead to an in vivo expression of an encoded gene product. One skilled in the art of molecular biology will recognize that the therapeutic element may include, but is not limited to a promoter sequence, a transgene, a poly A sequence, or a 3′ or 5′ UTR.

The term “transfects” as used herein refers to introduction of a nucleic acid into a eukaryotic cell. In some embodiments, the cell is not a plant tissue or a yeast cell.

The term “vector” as used herein refers to any vehicle that delivers a nucleic acid into a cell or organism. Examples include plasmid vectors, viral vectors, liposomes, or cationic lipids.

The term “viral backbone” as used herein refers to a nucleic acid sequence that does not contain a promoter, a gene, and a 3′ poly A signal or an untranslated region, but contain elements including, but not limited at site-specific genomic integration Rep and inverted terminal repeats (“ITRs”) or the binding site for the tRNA primer for reverse transcription, or a nucleic acid sequence component that induces a viral immunogenicity response when inserted in vivo, allows integration, affects specificity and activity of tissue specific promoters, causes transcriptional silencing or poses safety risks to the subject.

The term “vascular pressure pulse” refers to a pulse of pressure from a large volume of liquid to facilitate uptake of a vector into a cell. A skilled artisan recognizes that the amount and duration of the vascular pressure pulse is dependent on the tissue, size, and overall health of the recipient animal, and furthermore knows how to determine such parameters empirically.

The term “vector” as used herein refers to a construction comprised of genetic material designed to direct transformation of a targeted cell by delivering a nucleic acid sequence into that cell. A vector may contain multiple genetic elements positionally and sequentially oriented with other necessary elements such that an included nucleic acid cassette can be transcribed and when necessary translated in the transfected cells. These elements are operatively linked. The term “expression vector” refers to a DNA plasmid that contains all of the information necessary to produce a recombinant protein in a heterologous cell.

Involuntary culling is a major economic problem in the farm animal industry. Examples of “involuntary” culling include: being crippled (poor feet and legs); persistent mastitis problems; non-breeders; and disease or death. One aspect of the current invention is a method of decreasing an involuntary cull rate in farm animals, wherein the involuntary cull results from infection, disease, morbidity, or mortality. The method generally comprises delivering into a tissue of the farm animals an isolated nucleic acid expression construct that encodes a growth-hormone-releasing-hormone (“GHRH”) or functional biological equivalent thereof. Specific embodiments of this invention encompass various modes of delivering into the tissue of the farm animals the isolated nucleic acid expression construct (e.g. an electroporation method, a viral vector, in conjunction with a carrier, by parenteral route, or a combination thereof). In a first preferred embodiment, the isolated nucleic acid expression construct is delivered via an electroporation method comprising: a) penetrating the tissue in the farm animal with a plurality of needle electrodes, wherein the plurality of needle electrodes are arranged in a spaced relationship; b) introducing the isolated nucleic acid expression construct into the tissue between the plurality of needle electrodes; and c) applying an electrical pulse to the plurality of needle electrodes. A second preferred embodiments includes the isolated nucleic acid expression construct being delivered in a single dose, and the single dose comprising a total of about a 2 mg of nucleic acid expression construct. Generally the isolated nucleic acid expression construct is delivered into a tissue of the farm animals comprising diploid cells (e.g. muscle cells). In a third specific embodiment the isolated nucleic acid expression construct used for transfection comprises a HV-GHRH plasmid (SEQ ID#11). Other specific embodiments utilize other nucleic acid expression constructs (e.g. an optimized pAV0204 bGHRH plasmid (SEQ ID#19); a TI-GHRH plasmid (SEQ ID#12); TV-GHRH Plasmid (SEQ ID#13); 15/27/28 GHRH plasmid (SEQ ID#14); pSP-wt-GHRH plasmid; an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28). In a fourth specific embodiment, the isolated nucleic acid expression construct further comprises, a transfection-facilitating polypeptide (e.g. a charged polypeptide, or poly-L-glutamate). After delivering the isolated nucleic acid expression construct into the tissues of the farm animals, expression of the encoded GHRH or functional biological equivalent thereof is initiated. The encoded GHRH comprises a biologically active polypeptide; and the encoded functional biological equivalent of GHRH is a polypeptide that has been engineered to contain a distinct amino acid sequence while simultaneously having similar or improved biologically activity when compared to the GHRH polypeptide. One embodiment of a specific encoded GHRH or functional biological equivalent thereof is of formula (SEQ ID No: 6). The farm animal comprises a food animal, or a work animal (e.g. a pig, cow, sheep, goat or chicken).

A second aspect of the current invention includes a method of improving a body condition score (“BCS”) in farm animals comprising: delivering into a tissue of the farm animals an isolated nucleic acid expression construct that encodes a growth-hormone-releasing-hormone (“GHRH”) or functional biological equivalent thereof; wherein the BSC is an aid used to evaluate an overall nutritional state of the farm animal. The method generally comprises delivering into a tissue of the farm animals an isolated nucleic acid expression construct that encodes a growth-hormone-releasing-hormone (“GHRH”) or functional biological equivalent thereof. Specific embodiments of the second aspect of this invention encompass various modes of delivering into the tissue of the farm animals the isolated nucleic acid expression construct (e.g. an electroporation method, a viral vector, in conjunction with a carrier, by parenteral route, or a combination thereof). In a fifth preferred embodiment, the isolated nucleic acid expression construct is delivered via an electroporation method comprising: a) penetrating the tissue in the farm animal with a plurality of needle electrodes, wherein the plurality of needle electrodes are arranged in a spaced relationship; b) introducing the isolated nucleic acid expression construct into the tissue between the plurality of needle electrodes; and c) applying an electrical pulse to the plurality of needle electrodes. A sixth preferred embodiments includes the isolated nucleic acid expression construct being delivered in a single dose, and the single dose comprising a total of about a 2 mg of nucleic acid expression construct. Generally the isolated nucleic acid expression construct is delivered into a tissue of the farm animals comprising diploid cells (e.g. muscle cells). In a seventh specific embodiment the isolated nucleic acid expression construct used for transfection comprises a HV-GHRH plasmid (SEQ ID#11). Other specific embodiments utilize other nucleic acid expression constructs (e.g. an optimized pAV0204 bGHRH plasmid (SEQ ID# 19); a TI-GHRH plasmid (SEQ ID#12); TV-GHRH Plasmid (SEQ ID#13); 15/27/28 GHRH plasmid (SEQ ID#14); pSP-wt-GHRH plasmid; an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28). In a eighth specific embodiment, the isolated nucleic acid expression construct further comprises, a transfection-facilitating polypeptide (e.g. a charged polypeptide, or poly-L-glutamate). After delivering the isolated nucleic acid expression construct into the tissues of the farm animals, expression of the encoded GHRH or functional biological equivalent thereof is initiated. The encoded GHRH comprises a biologically active polypeptide; and the encoded functional biological equivalent of GHRH is a polypeptide that has been engineered to contain a distinct amino acid sequence while simultaneously having similar or improved biologically activity when compared to the GHRH polypeptide. One embodiment of a specific encoded GHRH or functional biological equivalent thereof is of formula (SEQ ID No: 6). The farm animal comprises a food animal, or a work animal (e.g. a pig, cow, sheep, goat or chicken).

A third aspect of the current invention includes a method of increasing milk production in a dairy cow comprising: delivering into muscle tissues of the dairy cow an isolated nucleic acid expression construct that encodes a growth-hormone-releasing-hormone (“GHRH”) or functional biological equivalent thereof. The method generally comprises delivering into a tissue of the dairy cow an isolated nucleic acid expression construct that encodes a growth-hormone-releasing-hormone (“GHRH”) or functional biological equivalent thereof. Specific embodiments of the third aspect of this invention encompass various modes of delivering into the tissue of the farm animals the isolated nucleic acid expression construct (e.g. an electroporation method, a viral vector, in conjunction with a carrier, by parenteral route, or a combination thereof). In a ninth preferred embodiment, the isolated nucleic acid expression construct is delivered via an electroporation method comprising: a) penetrating the tissue in the farm animal with a plurality of needle electrodes, wherein the plurality of needle electrodes are arranged in a spaced relationship; b) introducing the isolated nucleic acid expression construct into the tissue between the plurality of needle electrodes; and c) applying an electrical pulse to the plurality of needle electrodes. A tenth preferred embodiments includes the isolated nucleic acid expression construct being delivered in a single dose, and the single dose comprising a total of about a 2 mg of nucleic acid expression construct. Generally the isolated nucleic acid expression construct is delivered into a muscle tissue of the dairy cow comprising diploid cells (e.g. muscle cells). In a eleventh specific embodiment the isolated nucleic acid expression construct used for transfection comprises a HV-GHRH plasmid (SEQ ID#11). Other specific embodiments utilize other nucleic acid expression constructs (e.g. an optimized pAV0204 bGHRH plasmid (SEQ ID#19); a TI-GHRH plasmid (SEQ ID#12); TV-GHRH Plasmid (SEQ ID#13); 15/27/28 GHRH plasmid (SEQ ID#14); pSP-wt-GHRH plasmid; an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28). In a twelfth specific embodiment, the isolated nucleic acid expression construct further comprises, a transfection-facilitating polypeptide (e.g. a charged polypeptide, or poly-L-glutamate). After delivering the isolated nucleic acid expression construct into the tissues of the farm animals, expression of the encoded GHRH or functional biological equivalent thereof is initiated. The encoded GHRH comprises a biologically active polypeptide; and the encoded functional biological equivalent of GHRH is a polypeptide that has been engineered to contain a distinct amino acid sequence while simultaneously having similar or improved biologically activity when compared to the GHRH polypeptide. One embodiment of a specific encoded GHRH or functional biological equivalent thereof is of formula (SEQID No: 6).

The current invention also pertains to methods useful for increasing animal welfare in an animal. The general method of this invention comprises treating a subject with plasmid mediated gene supplementation. The method comprises delivering an isolated nucleic acid expression construct that encodes a growth-hormone-releasing-hormone (“GHRH”) or functional biological equivalent thereof into a tissue, such as a muscle, of the subject. Specific embodiments of this invention are directed toward decreasing culling rate and increasing body condition scores in treated animals, increasing milk production and enhancing immune function in treated animals. The subsequent in vivo expression of the GHRH or biological equivalent in the subject is sufficient to enhance welfare. It is also possible to enhance this method by placing a plurality of electrodes in a selected tissue, then delivering nucleic acid expression construct to the selected tissue in an area that interposes the plurality of electrodes, and applying a cell-transfecting pulse (e.g. electrical) to the selected tissue in an area of the selected tissue where the isolated nucleic acid expression construct was delivered. However, the cell-transfecting pulse need not be an electrical pulse, a vascular pressure pulse can also be utilized. Electroporation, direct injection, gene gun, or gold particle bombardment are also used in specific embodiments to deliver the isolated nucleic acid expression construct encoding the GHRH or biological equivalent into the subject. The subject in this invention comprises an animal (e.g. a human, a pig, a horse, a cow, a mouse, a rat, a monkey, a sheep, a goat, a dog, or a cat).

Recombinant GH replacement therapy is widely used in agriculture and clinically, with beneficial effects, but generally, the doses are supraphysiological. Such elevated doses of recombinant GH are associated with deleterious side-effects, for example, up to 30% of the recombinant GH treated subjects develop at a higher frequency insulin resistance (Gopinath and Etherton, 1989a; Gopinath and Etherton, 1989b; Verhelst et al., 1997) or accelerated bone epiphysis growth and closure in pediatric patients (Blethen and Rundle, 1996). In addition, molecular heterogeneity of circulating GH may have important implications in growth and homeostasis, which can lead to a less potent GH that has a reduced ability to stimulate the prolactin receptor (Satozawa et al., 2000; Tsunekawa et al., 1999; Wada et al., 1998). This effect is particularly inconvenient in milk-producing animals. These unwanted side effects result from the fact that treatment with recombinant exogenous GH protein raises basal levels of GH and abolishes the natural episodic pulses of GH. In contradistinction, no side effects have been reported for recombinant GHRH therapies. The normal levels of GHRH in the pituitary portal circulation range from about 150-to-800 pg/ml, while systemic circulating values of the hormone are up to about 100-500 pg/ml. Some patients with acromegaly caused by extracranial tumors have level that is nearly 10 times as high (e.g. 50 ng/ml of immunoreactive GHRH) (Thorner et al., 1984). Long-term studies using recombinant GHRH therapies (1-5 years) in children and elderly humans have shown an absence of the classical GH side-effects, such as changes in fasting glucose concentration or, in pediatric patients, the accelerated bone epiphysal growth and closure or slipping of the capital femoral epiphysis (Chevalier et al., 2000) (Duck et al., 1992; Vittone et al., 1997). Numerous studies in humans, sheep or pigs showed that continuous infusion with recombinant GHRH protein restores the normal GH pattern without desensitizing GHRH receptors or depleting GH supplies (Dubreuil et al., 1990). As this system is capable of a degree of feed-back which is abolished in the GH therapies, GHRH recombinant protein therapy may be more physiological than GH therapy. However, due to the short half-life of GHRH in vivo, frequent (one to three times per day) intravenous, subcutaneous or intranasal (requiring 300-fold higher dose) administrations are necessary (Evans et al., 1985; Thorner et al., 1986). Thus, as a chronic therapy, recombinant GHRH protein administration is not practical. A gene transfer approach, however could overcome this limitations to GHRH use. Moreover, a wide range of doses can be therapeutic. The choice of GHRH for a gene therapeutic application is favored by the fact that the gene, cDNA and native and several mutated molecules have been characterized for the pig, cattle and other species (Bohlen et al., 1983; Guillemin et al., 1982), and the measurement of therapeutic efficacy is straightforward and unequivocal.

Among the non-viral techniques for gene transfer in vivo, the direct injection of plasmid DNA into muscle is simple, inexpensive, and safe. The inefficient DNA uptake into muscle fibers after simple direct injection hag led to relatively low expression levels (Prentice et al., 1994; Wells et al., 1997) In addition, the duration of the transgene expression has been short (Wolff et al., 1990). The most successful previous clinical applications have been confined to vaccines (Danko and Wolff, 1994; Tsurumi et al., 1996). Recently, significant progress to enhance plasmid delivery in vivo and subsequently to achieve physiological levels of a secreted protein was obtained using the electroporation technique. Recently, significant progress has been obtained using electroporation to enhance plasmid delivery in vivo. Electroporation has been used very successfully to transfect tumor cells after injection of plasmid (Lucas et al., 2002; Matsubara et al., 2001) or to deliver the anti-tumor drug bleomycin to cutaneous and subcutaneous tumors in humans (Gehl et al., 1998; Heller et al., 1996). Electroporation also has been extensively used in mice (Lesbordes et al., 2002; Lucas et al., 2001; Vilquin et al., 2001), rats (Terada et al., 2001; Yasui et al., 2001), and dogs (Fewell et al., 2001) to deliver therapeutic genes that encode for a variety of hormones, cytokines or enzymes. Our previous studies using growth hormone releasing hormone (GHRH) showed that plasmid therapy with electroporation is scalable and represents a promising approach to induce production and regulated secretion of proteins in large animals and humans (Draghia-Akli et al., 1999; Draghia-Akli et al., 2002b). Electroporation also has been extensively used in rodents and other small animals (Bettan et al., 2000; Yin and Tang, 2001). It has been observed that the electrode configuration affects the electric field distribution, and subsequent results (Gehl et al., 1999; Miklavcic et al., 1998). Preliminary experiments indicated that for a large animal model, needle electrodes give consistently better reproducible results than external caliper electrodes.

The ability of electroporation to enhance plasmid uptake into the skeletal muscle has been well documented, as described above. In addition, plasmid formulated with PLG or polyvinylpyrrolidone (“PVP”) has been observed to increase gene transfection and consequently gene expression to up to 10 fold in the skeletal muscle of mice, rats and dogs (Fewell et al., 2001; Mumper et al., 1998). Although not wanting to be bound by theory, PLG will increase the transfection of the plasmid during the electroporation process, not only by stabilizing the plasmid DNA, and facilitating the intracellular transport through the membrane pores, but also through an active mechanism. For example, positively charged surface proteins on the cells could complex the negatively charged PLG linked to plasmid DNA through protein-protein interactions. When an electric field is applied, the surface proteins reverse direction and actively internalize the DNA molecules, process that substantially increases the transfection efficiency.

The plasmid supplementation approach to enhance animal welfare, decrease culling rates, and increase body condition scores described herein offers advantages over the limitations of directly injecting recombinant GH or GHRH protein. Expression of novel biological equivalents of GHRH that are serum protease resistant can be directed by an expression plasmid controlled by a synthetic muscle-specific promoter. Expression of such GHRH or biological equivalent thereof elicited high GH and IGF-I levels in subjects that have had the encoding sequences delivered into the cells of the subject by intramuscular injection and in vivo electroporation. Although in vivo electroporation is the preferred method of introducing the heterologous nucleic acid encoding system into the cells of the subject, other methods exist and should be known by a person skilled in the art (e.g. electroporation, lipofectamine, calcium phosphate, ex vivo transformation, direct injection, DEAE dextran, sonication loading, receptor mediated transfection, microprojectile bombardment, etc.). For example, it may also be possible to introduce the nucleic acid sequence that encodes the GHRH or functional biological equivalent thereof directly into the cells of the subject by first removing the cells from the body of the subject or donor, maintaining the cells in culture, then introducing the nucleic acid encoding system by a variety of methods (e.g. electroporation, lipofectamine, calcium phosphate, ex vivo transformation, direct injection, DEAE dextran, sonication loading, receptor mediated transfection, microprojectile bombardment, etc.), and finally reintroducing the modified cells into the original subject or other host subject (the ex vivo method). The GHRH sequence can be cloned into an adenovirus vector or an adeno-associated vector and delivered by simple intramuscular injection, or intravenously or intra-arterially. Plasmid DNA carrying the GHRH sequence can be complexed with cationic lipids or liposomes and delivered intramuscularly, intravenously or subcutaneous.

Administration as used herein refers to the route of introduction of a vector or carrier of DNA into the body. Administration can be directly to a target tissue or by targeted delivery to the target tissue after systemic administration. In particular, the present invention can be used for treating disease by administration of the vector to the body in order to establishing controlled expression of any specific nucleic acid sequence within tissues at certain levels that are useful for plasmid mediated supplementation. The preferred means for administration of vector and use of formulations for delivery are described above.

Muscle cells have the unique ability to take up DNA from the extracellular space after simple injection of DNA particles as a solution, suspension, or colloid into the muscle. Expression of DNA by this method can be sustained for several months. DNA uptake in muscle cells is further enhance utilizing in vivo electroporation.

Delivery of formulated DNA vectors involves incorporating DNA into macromolecular complexes that undergo endocytosis by the target cell. Such complexes may include lipids, proteins, carbohydrates, synthetic organic compounds, or inorganic compounds. The characteristics of the complex formed with the vector (size, charge, surface characteristics, composition) determine the bioavailability of the vector within the body. Other elements of the formulation function as ligands that interact with specific receptors on the surface or interior of the cell. Other elements of the formulation function to enhance entry into the cell, release from the endosome, and entry into the nucleus.

Delivery can also be through use of DNA transporters. DNA transporters refer to molecules which bind to DNA vectors and are capable of being taken up by epidermal cells. DNA transporters contain a molecular complex capable of non-covalently binding to DNA and efficiently transporting the DNA through the cell membrane. It is preferable that the transporter also transport the DNA through the nuclear membrane. See, e.g., the following applications all of which (including drawings) are hereby incorporated by reference herein: (1) Woo et al., U.S. Pat. No. 6,150,168 entitled: “A DNA Transporter System and Method of Use;” (2) Woo et al., PCT/US93/02725, entitled “A DNA Transporter System and method of Use”, filed Mar. 19, 1993; (3) Woo et al., U.S. Pat. No. 6,177,554 “Nucleic Acid Transporter Systems and Methods of Use;” (4) Szoka et al., U.S. Pat. No. 5,955,365 entitled “Self-Assembling Polynucleotide Delivery System;” and (5) Szoka et al., PCT/US93/03406, entitled “Self-Assembling Polynucleotide Delivery System”, filed Apr. 5, 1993.

Another method of delivery involves a DNA transporter system. The DNA transporter system consists of particles containing several elements that are independently and non-covalently bound to DNA. Each element consists of a ligand which recognizes specific receptors or other functional groups such as a protein complexed with a cationic group that binds to DNA. Examples of cations which may be used are spermine, spermine derivatives, histone, cationic peptides and/or polylysine; one element is capable of binding both to the DNA vector and to a cell surface receptor on the target cell. Examples of such elements are organic compounds which interact with the asialoglycoprotein receptor, the folate receptor, the mannose-6-phosphate receptor, or the carnitine receptor. A second element is capable of binding both to the DNA vector and to a receptor on the nuclear membrane. The nuclear ligand is capable of recognizing and transporting a transporter system through a nuclear membrane. An example of such ligand is the nuclear targeting sequence from SV40 large T antigen or histone. A third element is capable of binding to both the DNA vector and to elements which induce episomal lysis. Examples include inactivated virus particles such as adenovirus, peptides related to influenza virus hemagglutinin, or the GALA peptide described in the Skoka patent cited above.

Administration may also involve lipids. The lipids may form liposomes which are hollow spherical vesicles composed of lipids arranged in unilamellar, bilamellar, or multilamellar fashion and an internal aqueous space for entrapping water soluble compounds, such as DNA, ranging in size from 0.05 to several microns in diameter. Lipids may be useful without forming liposomes. Specific examples include the use of cationic lipids and complexes containing DOPE which interact with DNA and with the membrane of the target cell to facilitate entry of DNA into the cell.

Gene delivery can also be performed by transplanting genetically engineered cells. For example, immature muscle cells called myoblasts may be used to carry genes into the muscle fibers. Myoblast genetically engineered to express recombinant human growth hormone can secrete the growth hormone into the animal's blood. Secretion of the incorporated gene can be sustained over periods up to 3 months.

Myoblasts eventually differentiate and fuse to existing muscle tissue. Because the cell is incorporated into an existing structure, it is not just tolerated but nurtured. Myoblasts can easily be obtained by taking muscle tissue from an individual who needs plasmid-mediated supplementation and the genetically engineered cells can also be easily put back with out causing damage to the patient's muscle. Similarly, keratinocytes may be used to delivery genes to tissues. Large numbers of keratinocytes can be generated by cultivation of a small biopsy. The cultures can be prepared as stratified sheets and when grafted to humans, generate epidermis which continues to improve in histotypic quality over many years. The keratinocytes are genetically engineered while in culture by transfecting the keratinocytes with the appropriate vector. Although keratinocytes are separated from the circulation by the basement membrane dividing the epidermis from the dermis, human keratinocytes secrete into circulation the protein produced.

Delivery may also involve the use of viral vectors. For example, an adenoviral vector may be constructed by replacing the E1 region of the virus genome with the vector elements described in this invention including promoter, 5′UTR, 3′UTR and nucleic acid cassette and introducing this recombinant genome into 293 cells which will package this gene into an infectious virus particle. Virus from this cell may then be used to infect tissue ex vivo or in vivo to introduce the vector into tissues leading to expression of the gene in the nucleic acid cassette.

Although not wanting to be bound by theory, it is believed that in order to provide an acceptable safety margin for the use of such heterologous nucleic acid sequences in humans, a regulated gene expression system is mandated to possess low levels of basal expression of GHRH, and still retain a high ability to induce. Thus, target gene expression can be regulated by incorporating molecular switch technology. The HV-GHRH or biological equivalent molecule displays a high degree of stability in serum, with a half-life of 6 hours, versus the natural GHRH, that has a 6-12 minutes half-life. Thus, by combining the powerful electroporation DNA delivery method with stable and regulable GHRH or biological equivalent encoded nucleic acid sequences, a therapy can be utilized that will enhance animal welfare, decrease culling rates and increase body condition scores.

Vectors

The term “vector” is used to refer to a carrier nucleic acid molecule into which a nucleic acid sequence can be inserted for introduction into a cell wherein, in some embodiments, it can be replicated. A nucleic acid sequence can be native to the animal, or it can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), linear DNA fragments, and artificial chromosomes (e.g., YACs), although in a preferred embodiment the vector contains substantially no viral sequences. One of skill in the art would be well equipped to construct a vector through standard recombinant techniques.

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operatively linked coding sequence in a particular host cell. In addition to control sequences that govern transcription and translation, vectors and expression vectors may contain nucleic acid sequences that serve other functions as well and are described infra.

Plasmid Vectors

In certain embodiments, a linear DNA fragment from a plasmid vector is contemplated for use to transfect a eukaryotic cell, particularly a mammalian cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins. A skilled artisan recognizes that any plasmid in the art may be modified for use in the methods of the present invention. In a specific embodiment, for example, a GHRH vector used for the therapeutic applications is derived from pBlueScript KS+ and has a kanamycin resistance gene.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (“GST”) soluble fusion proteins for later purification and separation or cleavage. Other suitable fusion proteins are those with α-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

Promoters and Enhancers

A promoter is a control sequence that is a region of a nucleic acid sequence at which initiation and rate of transcription of a gene product are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter maybe one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant, synthetic or heterologous promoter, which refers to a promoter that is not normally associated with a nucleic acid sequence in its natural environment. A recombinant, synthetic or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Tables 2 and 3 list non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 2
Promoter and/or Enhancer

| Promoter/Enhancer | Relevant References |
|---|---|
| β-Actin | (Kawamoto et al., 1988; Kawamoto et al., 1989) |
| Muscle Creatine Kinase (MCK) | (Horlick and Benfield, 1989; Jaynes et al., 1988) |
| Metallothionein (MTII) | (Inouye et al., 1994; Narum et al., 2001; Skroch et al., |
| | 1993) |
| Albumin | (Pinkert et al., 1987; Tronche et al., 1989) |
| β-Globin | (Tronche et al., 1990; Trudel and Costantini, 1987) |
| Insulin | (German et al., 1995; Ohlsson et al., 1991) |
| Rat Growth Hormone | (Larsen et al., 1986) |
| Troponin I (TN I) | (Lin et al., 1991; Yutzey and Konieczny, 1992) |
| Platelet-Derived Growth Factor | (Pech et al., 1989) |
| (PDGF) | |
| Duchenne Muscular Dystrophy | (Klamut et al., 1990; Klamut et al., 1996) |
| Cytomegalovirus (CMV) | (Boshart et al., 1985; Dorsch-Hasler et al., 1985) |
| Synthetic muscle specific promoters | (Draghia-Akli et al., 1999; Draghia-Akli et al., 2002b; Li et |
| (c5-12, c1-28) | al., 1999) |

TABLE 3
Element/Inducer

| Element | Inducer |
|---|---|
| MT II | Phorbol Ester (TFA) |
| | Heavy metals |
| MMTV (mouse mammary tumor | Glucocorticoids |
| virus) | |
| β-Interferon | Poly(rI)x/Poly(rc) |
| Adenovirus 5 E2 | ElA |
| Collagenase | Phorbol Ester (TPA) |
| Stromelysin | Phorbol Ester (TPA) |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2κb | Interferon |
| HSP70 | ElA, SV40 Large T Antigen |
| Proliferin | Phorbol Ester-TPA |
| Tumor Necrosis Factor α | PMA |
| Thyroid Stimulating Hormone α | Thyroid Hormone |
| Gene | |

The identity of tissue-specific promoters or elements, as well as assays to characterize their activity, is well known to those of skill in the art. Nonlimiting examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Liu et al., 2000; Tsumaki et al., 1998), DIA dopamine receptor gene (Lee et al., 1997), insulin-like growth factor II (Dai et al., 2001; Wu et al., 1997), and human platelet endothelial cell adhesion molecule-1 (Almendro et al., 1996).

In a preferred embodiment, a synthetic muscle promoter is utilized, such as SPc5-12 (Li et al., 1999), which contains a proximal serum response element (“SRE”) from skeletal α-actin, multiple MEF-2 sites, MEF-1 sites, and TEF-1 binding sites, and greatly exceeds the transcriptional potencies of natural myogenic promoters. The uniqueness of such a synthetic promoter is a significant improvement over, for instance, issued patents concerning a myogenic promoter and its use (e.g. U.S. Pat. No. 5,374,544) or systems for myogenic expression of a nucleic acid sequence (e.g. U.S. Pat. No. 5,298,422). In a preferred embodiment, the promoter utilized in the invention does not get shut off or reduced in activity significantly by endogenous cellular machinery or factors. Other elements, including trans-acting factor binding sites and enhancers may be used in accordance with this embodiment of the invention. In an alternative embodiment, a natural myogenic promoter is utilized, and a skilled artisan is aware how to obtain such promoter sequences from databases including the National Center for Biotechnology Information (“NCBI”) GenBank database or the NCBI PubMed site. A skilled artisan is aware that these databases may be utilized to obtain sequences or relevant literature related to the present invention. INITIATION SIGNALS AND INTERNAL RIBOSOME BINDING SITES

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (“IRES”) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

Multiple Cloning Sites

Vectors can include a MCS, which is a nucleic acid region that contains multiple restriction enzyme sites, any of which can be used in conjunction with standard recombinant technology to digest the vector (see, for example, (Carbonelli et al., 1999; Cocea, 1997; Levenson et al., 1998) incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, (Chandler et al., 1997).

Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (“polyA”) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal, skeletal alpha actin 3′UTR or the human or bovine growth hormone polyadenylation signal, convenient and known to function well in various target cells. Polyadenylation may increase the stability of the transcript or may facilitate cytoplasmic transport.

Origins of Replication

In order to propagate a vector in a host cell, it may contain one or more origins of replication sites (often termed “ori”), which is a specific nucleic acid sequence at which replication is initiated. Alternatively an autonomously replicating sequence (“ARS”) can be employed if the host cell is yeast.

Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (“tk”) or chloramphenicol acetyltransferase (“CAT”) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

Mutagenesis

Where employed, mutagenesis was accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

Site-Directed Mutagenesis

Structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multi-residue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis. Other methods of site-directed mutagenesis are disclosed in U.S. Pat. Nos. 5,220,007; 5,284,760; 5,354,670; 5,366,878; 5,389,514; 5,635,377; and 5,789,166.

Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding and other methods known in the art.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

The underlying phenomenon of electroporation is believed to be the same in all cases, but the exact mechanism responsible for the observed effects has not been elucidated. Although not wanting to be bound by theory, the overt manifestation of the electroporative effect is that cell membranes become transiently permeable to large molecules, after the cells have been exposed to electric pulses. There are conduits through cell walls, which under normal circumstances, maintain a resting transmembrane potential of ca. 90 mV by allowing bi-directional ionic migration.

Although not wanting to be bound by theory, electroporation makes use of the same structures, by forcing a high ionic flux through these structures and opening or enlarging the conduits. In prior art, metallic electrodes are placed in contact with tissues and predetermined voltages, proportional to the distance between the electrodes are imposed on them. The protocols used for electroporation are defined in terms of the resulting field intensities, according to the formula E=V/d, where (“E”) is the field, (“V”) is the imposed voltage and (“d”) is the distance between the electrodes.

The electric field intensity E has been a very important value in prior art when formulating electroporation protocols for the delivery of a drug or macromolecule into the cell of the subject. Accordingly, it is possible to calculate any electric field intensity for a variety of protocols by applying a pulse of predetermined voltage that is proportional to the distance between electrodes. However, a caveat is that an electric field can be generated in a tissue with insulated electrodes (i.e. flow of ions is not necessary to create an electric field). Although not wanting to be bound by theory, it is the current that is necessary for successful electroporation not electric field per se.

During electroporation, the heat produced is the product of the interelectrode impedance, the square of the current, and the pulse duration. Heat is produced during electroporation in tissues and can be derived as the product of the inter-electrode current, voltage and pulse duration. The protocols currently described for electroporation are defined in terms of the resulting field intensities E, which are dependent on short voltage pulses of unknown current. Accordingly, the resistance or heat generated in a tissue cannot be determined, which leads to varied success with different pulsed voltage electroporation protocols with predetermined voltages. The ability to limit heating of cells across electrodes can increase the effectiveness of any given electroporation voltage pulsing protocol. For example, prior art teaches the utilization of an array of six needle electrodes utilizing a predetermined voltage pulse across opposing electrode pairs. This situation sets up a centralized pattern during an electroporation event in an area where congruent and intersecting overlap points develop. Excessive heating of cells and tissue along electroporation path will kill the cells, and limit the effectiveness of the protocol. However, symmetrically arranged needle electrodes without opposing pairs can produce a decentralized pattern during an electroporation event in an area where no congruent electroporation overlap points can develop.

Controlling the current flow between electrodes allows one to determine the relative heating of cells. Thus, it is the current that determines the subsequent effectiveness of any given pulsing protocol, and not the voltage across the electrodes. Predetermined voltages do not produce predetermined currents, and prior art does not provide a means to determine the exact dosage of current, which limits the usefulness of the technique. Thus, controlling an maintaining the current in the tissue between two electrodes under a threshold will allow one to vary the pulse conditions, reduce cell heating, create less cell death, and incorporate macromolecules into cells more efficiently when compared to predetermined voltage pulses.

Overcoming the above problem by providing a means to effectively control the dosage of electricity delivered to the cells in the inter-electrode space by precisely controlling the ionic flux that impinges on the conduits in the cell membranes. The precise dosage of electricity to tissues can be calculated as the product of the current level, the pulse length and the number of pulses delivered. Thus, a specific embodiment of the present invention can deliver the electroporative current to a volume of tissue along a plurality of paths without, causing excessive concentration of cumulative current in any one location, thereby avoiding cell death owing to overheating of the tissue.

Although not wanting to be bound by theory, the nature of the voltage pulse to be generated is determine by the nature of tissue, the size of the selected tissue and distance between electrodes. It is desirable that the voltage pulse be as homogenous as possible and of the correct amplitude. Excessive field strength results in the lysing of cells, whereas a low field strength results in reduced efficacy of electroporation. Some electroporation devices utilize the distance between electrodes to calculate the electric field strength and predetermined voltage pulses for electroporation. This reliance on knowing the distance between electrodes is a limitation to the design of electrodes. Because the programmable current pulse controller will determine the impedance in a volume of tissue between two electrodes, the distance between electrodes is not a critical factor for determining the appropriate electrical current pulse. Therefore, an alternative embodiment of a needle electrode array design would be one that is non-symmetrical. In addition, one skilled in the art can imagine any number of suitable symmetrical and non-symmetrical needle electrode arrays that do not deviate from the spirit and scope of the invention. The depth of each individual electrode within an array and in the desired tissue could be varied with comparable results. In addition, multiple injection sites for the macromolecules could be added to the needle electrode array.

Restriction Enzymes

In some embodiments of the present invention, a linear DNA fragment is generated by restriction enzyme digestion of a parent DNA molecule. Examples of restriction enzymes are provided below.

| | |
|---|---|
| Name | Recognition Sequence |

| | |
|---|---|
| AatII | GACGTC |
| Acc65 I | GGTACC |
| Acc I | GTMKAC |
| Aci I | CCGC |
| Acl I | AACGTT |
| Afe I | AGCGCT |
| Afl II | CTTAAG |
| Afl III | ACRYGT |
| Age I | ACCGGT |
| Ahd I | GACNNNNNGTC |
| Alu I | AGCT |
| Alw I | GGATC |
| AlwN I | CAGNNNCTG |
| Apa I | GGGCCC |
| ApaL I | GTGCAC |
| Apo I | RAATTY |
| Asc I | GGCGCGCC |
| Ase I | ATTAAT |
| Ava I | CYCGRG |
| Ava II | GGWCC |
| Avr II | CCTAGG |
| Bae I | NACNNNNGTAPyCN |
| BamH I | GGATCC |
| Ban I | GGYRCC |
| Ban II | GRGCYC |
| Bbs I | GAAGAC |
| Bbv I | GCAGC |
| BbvC I | CCTCAGC |
| Bcg I | CGANNNNNNTGC |
| BciV I | GTATCC |
| Bcl I | TGATCA |
| Bfa I | CTAG |
| Bgl I | GCCNNNNNGGC |
| Bgl II | AGATCT |
| Blp I | GCTNAGC |
| Bmr I | ACTGGG |
| Bpm I | CTGGAG |
| BsaA I | YACGTR |
| BsaB I | GATNNNNATC |
| BsaH I | GRCGYC |
| Bsa I | GGTCTC |
| BsaJ I | CCNNGG |
| BsaW I | WCCGGW |
| BseR I | GAGGAG |
| Bsg I | GTGCAG |
| BsiE I | CGRYCG |
| BsiHKA I | GWGCWC |
| BsiW I | CGTACG |
| Bsl I | CCNNNNNNNGG |
| BsmA I | GTCTC |
| BsmB I | CGTCTC |
| BsmF I | GGGAC |
| Bsm I | GAATGC |
| BsoB I | CYCGRG |
| Bsp1286 I | GDGCHC |
| BspD I | ATCGAT |
| BspE I | TCCGGA |
| BspH I | TCATGA |
| BspM I | ACCTGC |
| BsrB I | CCGCTC |
| BsrD I | GCAATG |
| BsrF I | RCCGGY |
| BsrG I | TGTACA |
| Bsr I | ACTGG |
| BssH II | GCGCGC |
| BssK I | CCNGG |
| Bst4C I | ACNGT |
| BssS I | CACGAG |
| BstAP I | GCANNNNNTGC |
| BstB I | TTCGAA |
| BstE II | GGTNACC |
| BstF5 I | GGATGNN |
| BstN I | CCWGG |
| BstU I | CGCG |
| BstX I | CCANNNNNNTGG |
| BstY I | RGATCY |
| BstZ17 I | GTATAC |
| Bsu36 I | CCTNAGG |
| Btg I | CCPuPyGG |
| Btr I | CACGTG |
| Cac8 I | GCNNGC |
| Cla I | ATCGAT |
| Dde I | CTNAG |
| Dpn I | GATC |
| Dpn II | GATC |
| Dra I | TTTAAA |
| Dra III | CACNNNGTG |
| Drd I | GACNNNNNNGTC |
| Eae I | YGGCCR |
| Eag I | CGGCCG |
| Ear I | CTCTTC |
| Eci I | GGCGGA |
| EcoN I | CCTNNNNNAGG |
| EcoO109 I | RGGNCCY |
| EcoR I | GAATTC |
| EcoR V | GATATC |
| Fau I | CCCGCNNNN |
| Fnu4H I | GCNGC |
| Fok I | GGATG |
| Fse I | GGCCGGCC |
| Fsp I | TGCGCA |
| Hae II | RGCGCY |
| Hae III | GGCC |
| Hga I | GACGC |
| Hha I | GCGC |
| Hinc II | GTYRAC |
| Hind III | AAGCTT |
| Hinf I | GANTC |
| HinP1 I | GCGC |
| Hpa I | GTTAAC |
| Hpa II | CCGG |
| Hph I | GGTGA |
| Kas I | GGCGCC |
| Kpn I | GGTACC |
| Mbo I | GATC |
| Mbo II | GAAGA |
| Mfe I | CAATTG |
| Mlu I | ACGCGT |
| Mly I | GAGTCNNNNN |
| Mnl I | CCTC |
| Msc I | TGGCCA |
| Mse I | TTAA |
| Msl I | CAYNNNNRTG |
| MspA1 I | CMGCKG |
| Msp I | CCGG |
| Mwo I | GCNNNNNNNGC |
| Nae I | GCCGGC |
| Nar I | GGCGCC |
| Nci I | CCSGG |
| Nco I | CCATGG |
| Nde I | CATATG |
| NgoMI V | GCCGGC |
| Nhe I | GCTAGC |
| Nla III | CATG |
| Nla IV | GGNNCC |
| Not I | GCGGCCGC |
| Nru I | TCGCGA |
| Nsi I | ATGCAT |
| Nsp I | RCATGY |
| Pac I | TTAATTAA |
| PaeR7 I | CTCGAG |
| Pci I | ACATGT |
| PflF I | GACNNNGTC |
| PflM I | CCANNNNNTGG |
| PleI | GAGTC |
| Pme I | GTTTAAAC |
| Pml I | CACGTG |
| PpuM I | RGGWCCY |
| PshA I | GACNNNNGTC |
| Psi I | TTATAA |
| PspG I | CCWGG |
| PspOM I | GGGCCC |
| Pst I | CTGCAG |
| Pvu I | CGATCG |
| Pvu II | CAGCTG |
| Rsa I | GTAC |
| Rsr II | CGGWCCG |
| Sac I | GAGCTC |
| Sac II | CCGCGG |
| Sal I | GTCGAC |
| Sap I | GCTCTTC |
| Sau3A I | GATC |
| Sau96 I | GGNCC |
| Sbf I | CCTGCAGG |
| Sca I | AGTACT |
| ScrF I | CCNGG |
| SexA I | ACCWGGT |
| SfaN I | GCATC |
| Sfc I | CTRYAG |
| Sfi I | GGCCNNNNNGGCC |
| Sfo I | GGCGCC |
| SgrA I | CRCCGGYG |
| Sma I | CCCGGG |
| Sml I | CTYRAG |
| SnaB I | TACGTA |
| Spe I | ACTAGT |
| Sph I | GCATGC |
| Ssp I | AATATT |
| Stu I | AGGCCT |
| Sty I | CCWWGG |
| Swa I | ATTTAAAT |
| Taq I | TCGA |
| Tfi I | GAWTC |
| Tli I | CTCGAG |
| Tse I | GCWGC |
| Tsp45 I | GTSAC |
| Tsp509 I | AATT |
| TspR I | CAGTG |
| Tth11I I | GACNNNGTC |
| Xba I | TCTAGA |
| Xcm I | CCANNNNNNNNNTGG |
| Xho I | CTCGAG |
| Xma I | CCCGGG |
| Xmn I | GAANNNNTTC |

The term “restriction enzyme digestion” of DNA as used herein refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffer solution. Appropriate buffers and substrate amounts for particular restriction enzymes are specified by the manufacturer. Restriction enzymes are used to ensure plasmid integrity and correctness.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Construction of DNA Vectors and Methods in Animal Subject

In order to decrease voluntary cull rates, increase milk production, and increase body condition scores by utilizing plasmid mediated gene supplementation, it was first necessary to design several GHRH constructs. Briefly, the plasmid vectors contained the muscle specific synthetic promoter SPc5-12 (SEQ ID#)(Li et al., 1999) attached to a wild type or analog porcine GHRH. The analog GHRH sequences were generated by site directed mutagenesis as described in methods section. Nucleic acid sequences encoding GHRH or analog were cloned into the BamHI/HindIII sites of pSPc5-12 plasmid, to generate pSP-GHRH (SEQ ID#15).

DNA constructs: Plasmid vectors containing the muscle specific synthetic promoter SPc5-12 (SEQ ID#7) were previously described (Li et al., 1999). Wild type and mutated porcine GHRH cDNA's were generated by site directed mutagenesis of GHRH cDNA (SEQ ID#9) (Altered Sites II in vitro Mutagenesis System, Promega, Madison, Wis.), and cloned into the BamHI/Hind III sites of pSPc5-12, to generate pSP-wt-GHRH (SEQ ID#15), or pSP-HV-GHRH (SEQ ID#11), respectively. The wild type porcine GHRH was obtained by sire directed mutagenesis of human GHRH cDNA (1-40)OH at positions 34: Ser to Arg, 38: Arg to Glu; the mutated porcine HV-GHRH DNA was obtained by site directed mutagenesis of porcine GHRH cDNA (1-40)OH at positions 1: Tyr to His, 2 Ala to Val, 15: Gly to Ala, 27: Met to Leu, 28: Ser to Asn, (Altered Sites II in vitro Mutagenesis System, Promega, Madison, Wis.), and cloned into the BamHI/Hind III sites of pSP-GHRH. The 3′ untranslated region (3′UTR) of growth hormone was cloned downstream of GHRH cDNA. The resultant plasmids contained mutated coding region for GHRH, and the resultant amino acid sequences were not naturally present in mammals. Although not wanting to be bound by theory, the enhanced welfare, decreased culling rate and increased body condition scores are determined ultimately by the circulating levels of mutated hormones. Several different plasmids that encoded different mutated amino acid sequences of GHRH or functional biological equivalent thereof are as follows:

| | |
|---|---|
| Plasmid | Encoded Amino Acid Sequence |

| | | |
|---|---|---|
| wt-GHRH | YADAIFTNSYRKVLGQLSARKLLQDIMSRQQGERNQEQGA-OH | (SEQ ID #10) |
| HV-GHRH | HVDAIFTNSYRKVLAQLSARKLLQDILNRQQGERNQEQGA-OH | (SEQ ID #11) |
| TI-GHRH | YIDAIFTNSYRKVLAQLSARKLLQDILNRQQGERNQEQGA-OH | (SEQ ID #12) |
| TV-GHRH | YVDAIFTNSYRKVLAQLSARKLLQDILNRQQGERNQEQGA-OH | (SEQ ID #13) |
| 15/27/28-GHRH | YADAIFTNSYRKVLAQLSARKLLQDILNRQQGERNQEQGA-OH | (SEQ ID #14) |

In general, the encoded GHRH or functional biological equivalent thereof is of formula:

—X₁—X₂-DAIFTNSYRKVL-X₃-QLSARKLLQDI-X₄—X₅-RQQGERNQEQGA-OH (SEQ ID#6)

wherein: X₁ is a D-or L-isomer of an amino acid selected from the group consisting of tyrosine (“Y”), or histidine (“H”); X₂ is a D-or L-isomer of an amino acid selected from the group consisting of alanine (“A”), valine (“V”), or isoleucine (“I”); X₃ is a D-or L-isomer of an amino acid selected from the group consisting of alanine (“A”) or glycine (“G”); X₄ is a D-or L-isomer of an amino acid selected from the group consisting of methionine (“M”), or leucine (“L”); X₅ is a D-or L-isomer of an amino acid selected from the group consisting of serine (“S”) or asparagines (“N”).

The plasmids described above do not contain polylinker, IGF-I gene, a skeletal alpha-actin promoter or a skeletal alpha actin 3′ UTR/NCR. Furthermore, these plasmids were introduced by muscle injection, followed by in vivo electroporation, as described below.

In terms of “functional biological equivalents”, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Functional biological equivalents are thus defined herein as those proteins (and polynucleotides) in selected amino acids (or codons) may be substituted. A peptide comprising a functional biological equivalent of GHRH is a polypeptide that has been engineered to contain distinct amino acid sequences while simultaneously having similar or improved biologically activity when compared to GHRH. For example one biological activity of GHRH is to facilitate growth hormone (“GH”) secretion in the subject.

Optimized Plasmid Backbone. One aspect of the current invention is the optimized plasmid backbone. The synthetic plasmids presented below contain eukaryotic sequences that are synthetically optimized for species specific mammalian transcription. An existing pSP-HV-GHRH plasmid (“pAV0125”) (SeqID#29), was synthetically optimized to form a new plasmid (“pAV0201”)(SeqID#30). The plasmid pAV0125 was described in U.S. patent application Ser. No. 09/624,268 filed on Jul. 24, 2000, 2000 and titled “Super Active Porcine Growth Hormone Releasing Hormone Analog” with Schwartz, et al., listed as inventors, (“the Schwartz '268 application”). This 3,534 bp plasmid pAV0125 (SeqID #29) contains a plasmid backbone with various component from different commercially available plasmids, for example, a synthetic promoter SPc5-12 (SeqID #7), a modified porcine GHRH sequence (SeqID #4), and a 3′end of human growth hormone (SeqID #8). Other examples of optimized synthetic plasmids include pAV0202 (SeqID #17), pAV0203 (SeqID #18), pAV0204 (SeqID #19), pAV0205 (SeqID #20), pAV0206 (SeqID #21), pAV0207 (SeqID #28). The therapeutic encoded gene for such optimized plasmids may also include optimized nucleic acid sequences that encode modified GHRH molecules or functional biological equivalents thereof.

### Example 2

One embodiment of this invention teaches that plasmid mediated gene supplementation of GHRH or a functional biological equivalent thereof, decreases the mortality rate of treated bovine heifers. For example thirty-two pregnant bovine heifers were treated with 2 mg pSP-HV-GHRH once during the last trimester of gestation and designated as the “treated” group. Similarly 20 pregnant bovine animals from same source did not receive plasmid treatment and served as controls. Plasmid treatment comprises endotoxin-free plasmid (Qiagen Inc., Chatsworth, Calif.) preparations of pSP-HV-GHRH that were diluted in water and formulated with PLG 0.01% (w/v). Dairy cows were given a total quantity of 2 mg pSP-HV-GHRH intramuscularly, into the neck muscles. The plasmid was injected directly into the muscle, using an 21 G needle (Becton-Dickinson, Franklin Lacks, N.J.). Two minutes after injection, the injected muscle was electroporated, 5 pulses, 1 Amp, 50 milliseconds/pulse, as described (Draghia-Akli et al., 2002a). In all injections the needles were completely inserted into the muscle.

The mortality rate for the heifers, the calves at birth, and the post-natal calves were recorded and summarized in FIG. 1. As shown in FIG. 1A, the mortality of treated heifers is 3% compared to 20% mortality in control heifers, which represents an 85% decrease in the mortality rate of treated heifers compared to controls. As shown in FIG. 1B, the mortality rate of calves born from treated heifers was 18.8%, and the mortality rate of calves born from control heifers was 25%. Accordingly, calves from treated heifers showed a 25% decrease in mortality at birth compared with calves born from non-treated heifer controls. The post natal survival of calves born from treated heifers was 0%, whereas calves born from control heifers represented 21.4%, as shown in FIG. 1C. Thus, a 100% decrease in mortality rate was observed in calves from treated heifers.

### Example 3

The same two groups of heifers described in Example 2 were further studied by comparing the body condition scores of the treated heifers and control heifers 60-80 days in milk (“DIM”). The body condition score (“BCS”) is an aid used to evaluate the overall nutrition and management of dairy heifers and cows. Condition scores range from 1 (very thin cow) to 5 (a severely over conditioned cow), with guidelines relating to condition score ranges at various stages of the production cycle. Cows are scored by both observing and handling the backbone, loin, and rump areas as these areas do not have a muscle tissue covering only skin and fat deposits (Rodenburg, 1996). BCS serves as management tool with respect to feeding, breeding, and recognition of health status in dairy herds. (Dechow et al., 2002; Domecq et al., 1997; Parker, 1996; Studer, 1998). Body condition is a reflection of the body fat reserves carried by the animal. These reserves can be used by the cow in periods when she is unable to eat enough to satisfy her energy needs. In dairy cows, this normally happens during early lactation, when the animals tend to be in a negative energy balance resulting in loss of body condition. The rule of thumb is that animals should not lose more than 1 BCS unit during the early lactation period.

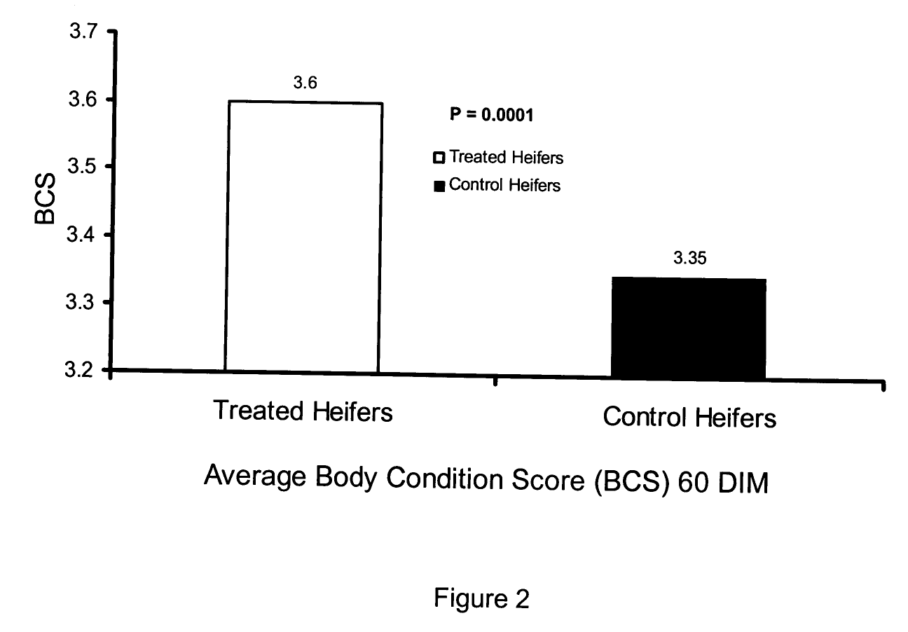

As shown in FIG. 2, the BCS in heifers treated with pSP-HV-GHRH versus controls at 60-80 days in milk (“DIM”) showed a statistically significant improvement having a BCS of 3.6 compared with a BCS of 3.35 for non-treated controls (p<0.0001). Although not wanting to be bound by theory, at 60 days in milk control animals show a significant decrease in body condition scores (“BCS”), which may be resultant of complex physiological mechanisms. Minimized BCS loss translates to decreased mobilization of body tissue, resulting in increased peak milk production and reduced breeding interval. Although not wanting to be bound by theory, these attributes could also result in savings in feed costs to bring the cow back to the appropriate BCS at “dry off” and calving.

### Example 4

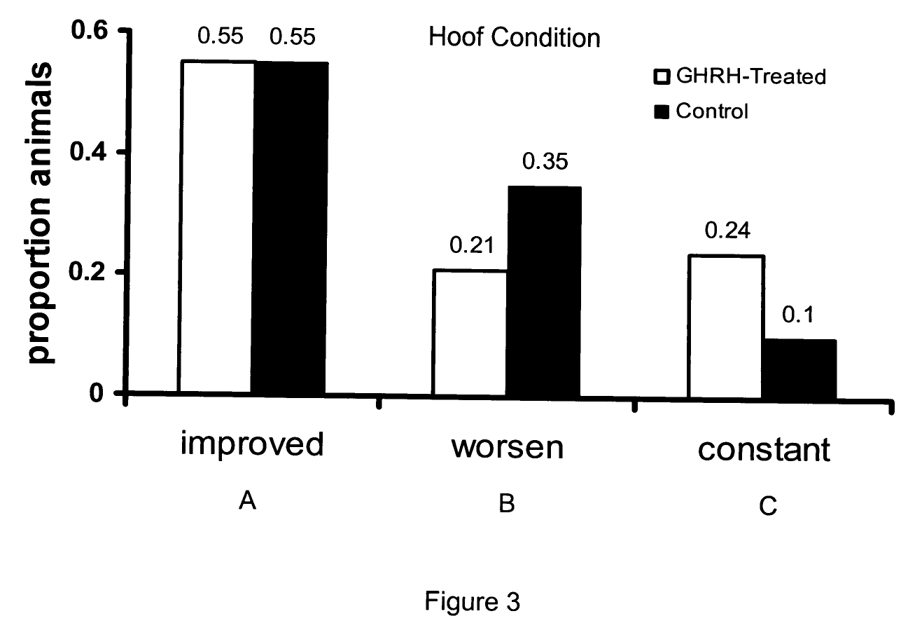

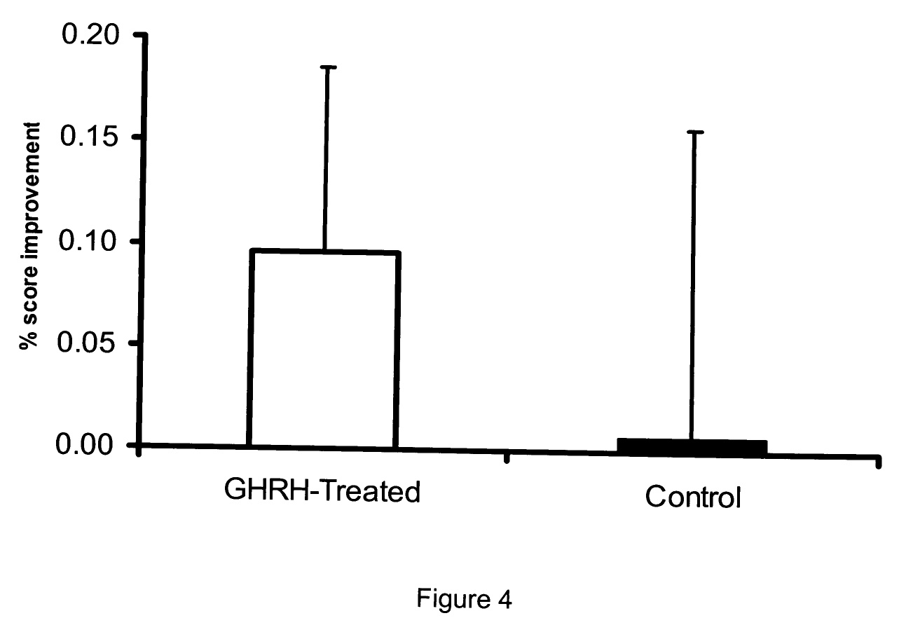

The same two groups of heifers described in Example 2 were further studied by comparing the percentage of cows with foot problems during the course of the study. Foot problems were also one of the principal causes of morbidity in these groups of animals. pSP-HV-GHRH treated and control animals with foot problems were divided into 3 groups: A) foot problems that improved; B) foot problems that became worse; and C) foot problems that remained constant. The proportions of animals that improved, became worse, or remained constant are shown in FIGS. 3A, 3B and 3C respectively. The proportion of animals that showed improved foot problems were not different between the pSP-HV-GHRH treated animals and control groups, as shown in FIG. 3A. In contrast, the proportion of control animals having foot problems worsen throughout the course of the study was 40% higher when compared to the treated animals, as shown in FIG. 3B. Similarly, the proportion of animals that neither improved nor became worse are shown in FIG. 3C. The overall hoof score improved during the course of the experiment in treated animals versus controls, as shown in FIG. 4. Although not wanting to be bound by theory, the results depicted in FIG. 4 were not significantly statistical due to high inter-animal variability in the control group.

### Example 5

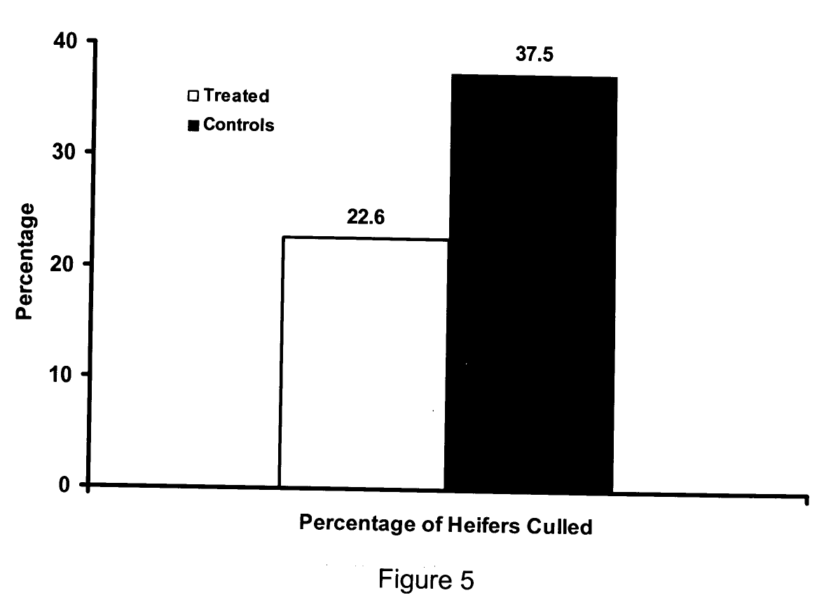

The same two groups of heifers described in Example 2 were further studied by determining the total percentage of involuntary culls in heifers treated with pSP-HV-GHRH versus controls at 120 days in milk, as shown in FIG. 5. The percentage of involuntary cull rates for treated animals was almost 40% lower when compared to non-treated controls.

### Example 6

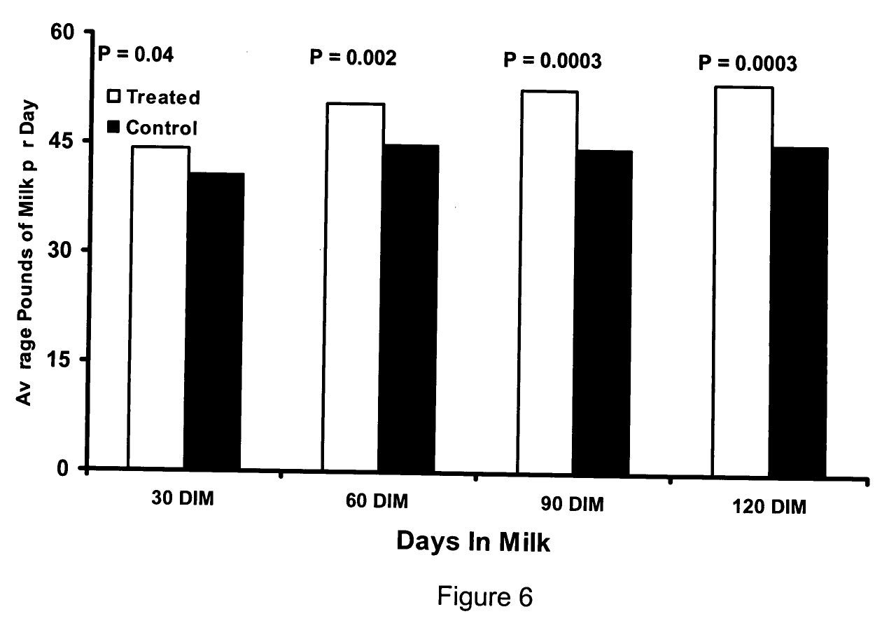

The same two groups of heifers described in Example 2 were further studied by determining the total milk production in animals treated with pSP-HV-GHRH versus controls at different time points (e.g. 30-120 days in milk (“DIM”)). As shown in FIG. 6, at all time points recorded, the pSP-HV-GHRH treated animals produced more pounds of milk per day when compared to non-treated controls. P value for each time point is also stated.

### Example 7

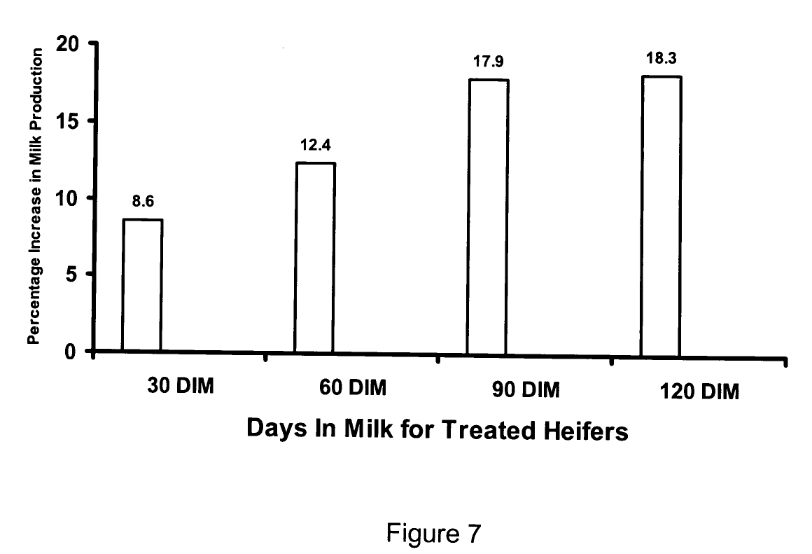

The same two groups of heifers described in Example 2 were further studied by determining the percentage of increased milk production in pSP-HV-GHRH treated cows versus controls at different time periods. As shown in FIG. 7, the percentage of milk production in the pSP-HV-GHRH treated heifers continually increases from 30 to 120 days in milk. The increase in animal welfare was also reflected in the milk production. At all recorded time points (30-120 DIM) treated animals produced more milk than controls (FIG. 6 and FIG. 7), wherein the p-value for each time point is statistically significant.

### Example 8

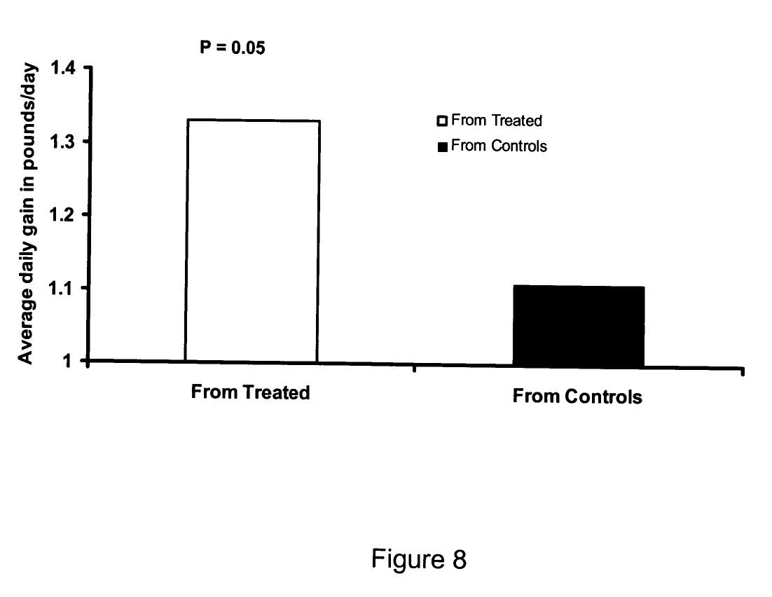

The same two groups of heifers described in Example 2 were further studied by comparing the average daily weight gains in calves born to treated heifers versus those born to control heifers. As shown in FIG. 8, the average daily weight gain in pounds was higher for calves from pSP-HV-GHRH treated heifers compared with calves from non-treated control heifers. Although not wanting to be bound by theory, it is known that treatment with recombinant GHRH given as injections 2 weeks prior to parturition increases weight of pigs at 13 days and at weaning and improves pig survival (Etienne et al., 1992). Nevertheless, in this previous case, the effect is not sustained for longer periods of time, as in our case.

### Example 9

Based upon the depicted benefits from the above examples, it is possible to derive an economic model based on the additional milk resulting from pSP-HV-GHRH treatment. The assumptions for this economic models is based upon 300 days in milk (“DIM”), minus additional feed costs for increased intake. As shown in FIG. 9A, the increase in annual income from additional milk production is additionally based upon a $110 per cow per year for a first and second parity cost of treatment. Chart values are show either 8 or 12 pounds of milk being produced per day per cow, and $0.12 or $0.14 per pound of milk per cow. Additionally values are computed for having either one or 350 cows producing at the indicated level of production (e.g. 8 or 12 pounds of milk per day) at the indicated price (e.g. $0.12 or $0.14 per pound of milk). FIG. 9B shows a cost of treatment for a first, second and third parity at $110/cow/year.

### Example 10

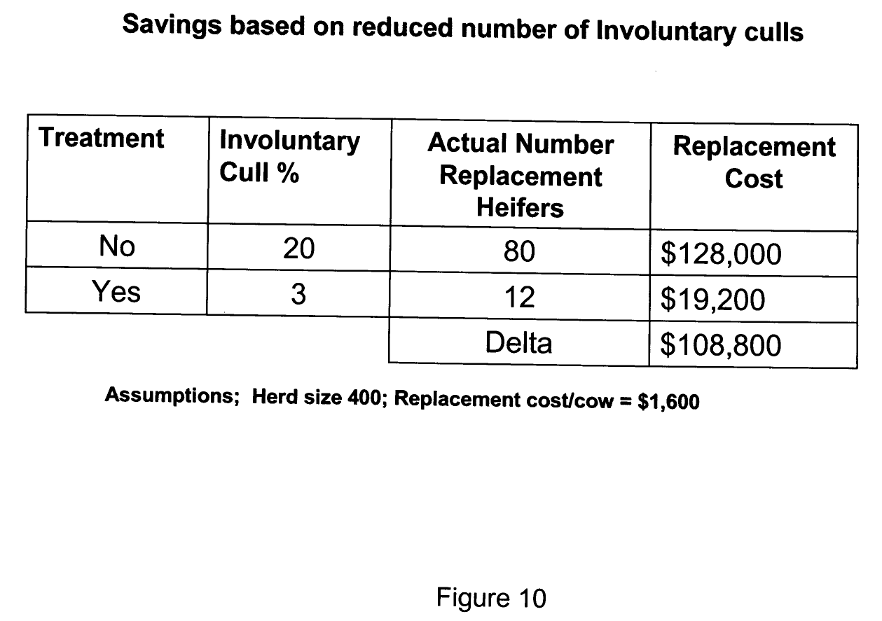

Based upon the depicted benefits from the above examples, it is possible to derive an economic model based upon the reduced number of involuntary culls. FIG. 10 shows how treating animals with pSP-HV-GHRH can result in a $108,000 savings on replacement cost, values based on assuming a herd size of 400, wherein the replacement cost of a single cow is $1,600.

### Example 11

One concern when treating animals with bST or GHRH is that the treatment will ultimately stimulate GH and IGF-I production resulting in residual hormones being present in the milk. Numerous studies targeting this issue were conducted at Monsanto, Inc. (Hammond et al., 1990), and the milk from cows treated with bST was found to be safe for consumption with a zero withdrawal time. This concern was addressed with eighteen cows that were divided into two groups. The animals were paired for parity and calving date. Nine cows were treated with plasmid mediated gene supplementation having a treatment of 2 mg pSP-HV-GHRH once during late lactation, this groups was denoted as the treated group. In addition, 9 cows from same source continued initially on a bST (bovine somatotropin, GH) regimen having one treatment every 14 days, this group was denoted as the control group. The control group was not given bST treatment after calving because the manufacturer instructions do not recommend that bST be given during the first 60 days of lactation. As shown below, IGF-I levels were evaluated at 14-28 days post-injection and daily average pounds of milk per day was measured after calving.

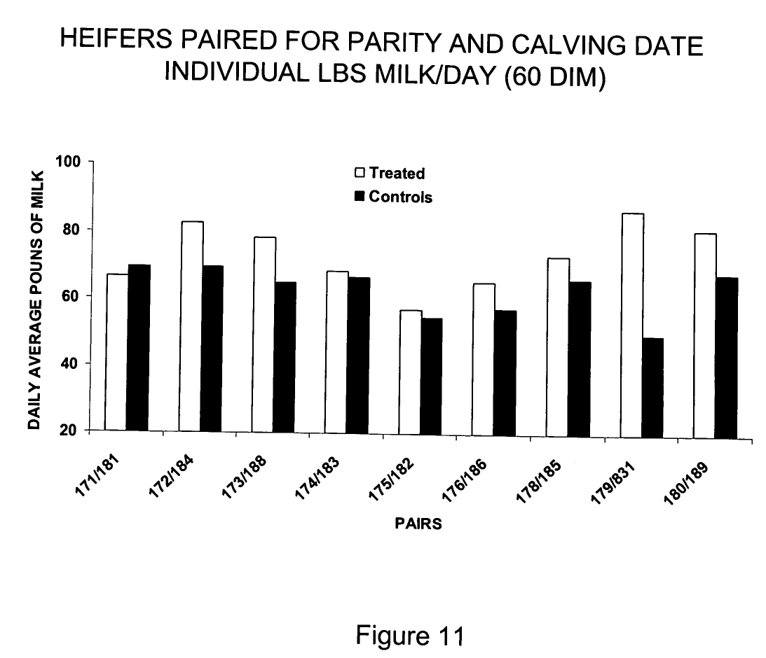

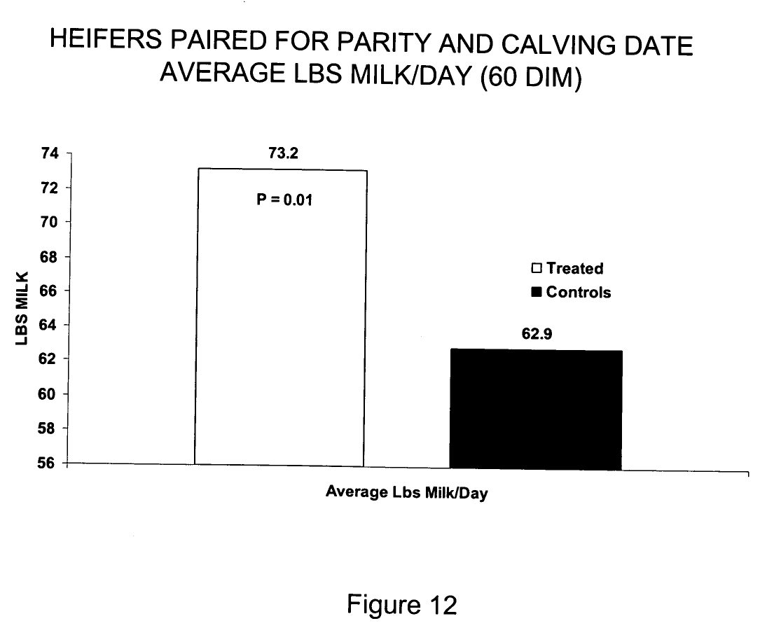

The daily average production of milk was determined for treated and control heifers paired for parity and calving date. As shown FIG. 11, the milk production for individual animals both treated and controls is compared. The data represents 60 days in milk, and in all but one pair, the animal treated with pSP-HV-GHRH had a higher milk production compared with controls. FIG. 12 show the average milk production in treated and control groups. FIG. 12 data represents animals at 60 DIM, and animals treated with pSP-HV-GHRH had a higher milk production than controls (P<0.01).

### Example 12

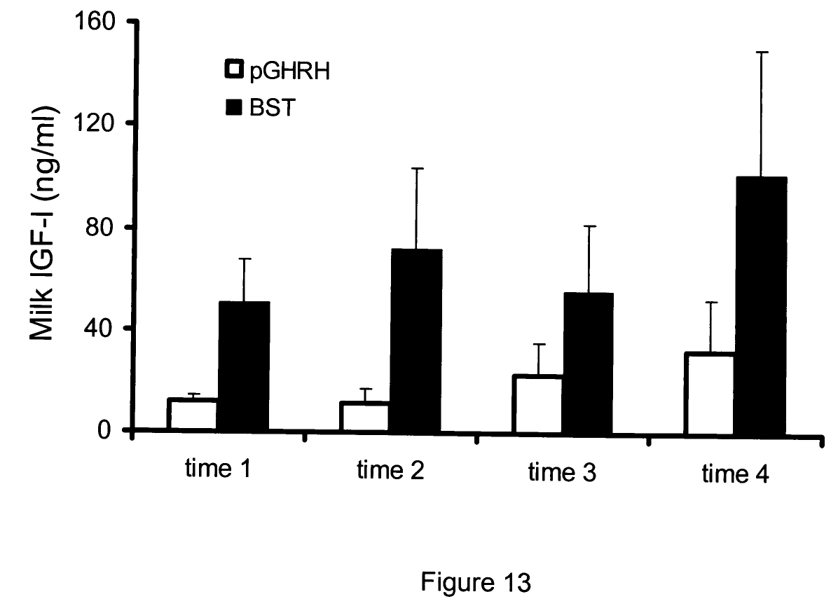

The same two groups of heifers described in Example 11 were further studied by assaying the average IGF-I levels in milk from treated and control groups. As shown in FIG. 13, IGF-I levels were determined at days 14-28 post treatment. The treated group represents 9 cows pGHRH-treated and controls are 9 bST-treated animals. The milk IGF-I levels were lower in pSP-HV-GHRH-treated animals (3-5 fold) at all time points tested. As illustrated in FIG. 13, Time 1=14 days post-treatment; Time 2=19 days post-treatment; Time 3=23 days post-treatment; Time 4=28 days post-treatment. All samples were assayed in triplicate.

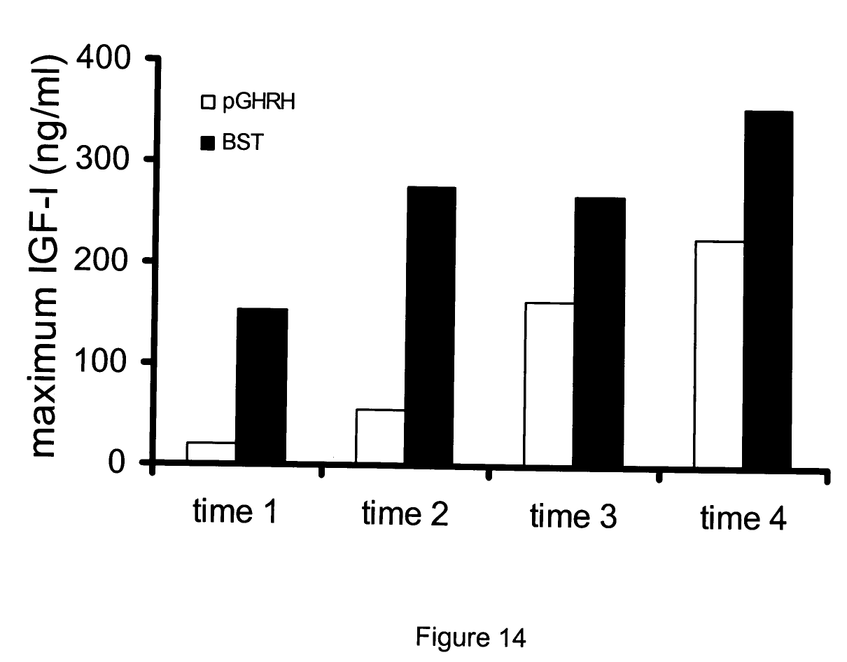

The maximum milk IGF-I levels from cows at days 14-28 post treatment are shown in FIG. 14. The two groups of animals were 9 pGHRH-treated and 9 bST-treated animals. Time 1=14 days post-treatment; Time 2=19 days post-treatment; Time 3=23 days post-treatment; Time 4=28 days post-treatment, as shown in FIG. 14. Maximum milk IGF-I levels were lower in pSP-HV-GHRH-treated animals at all time points tested.

### Example 13

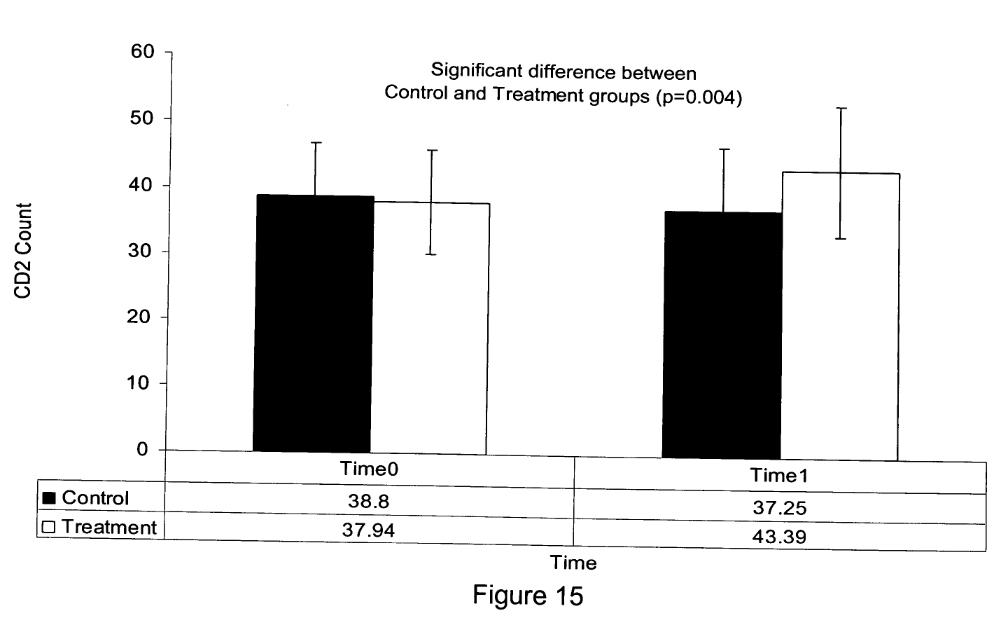

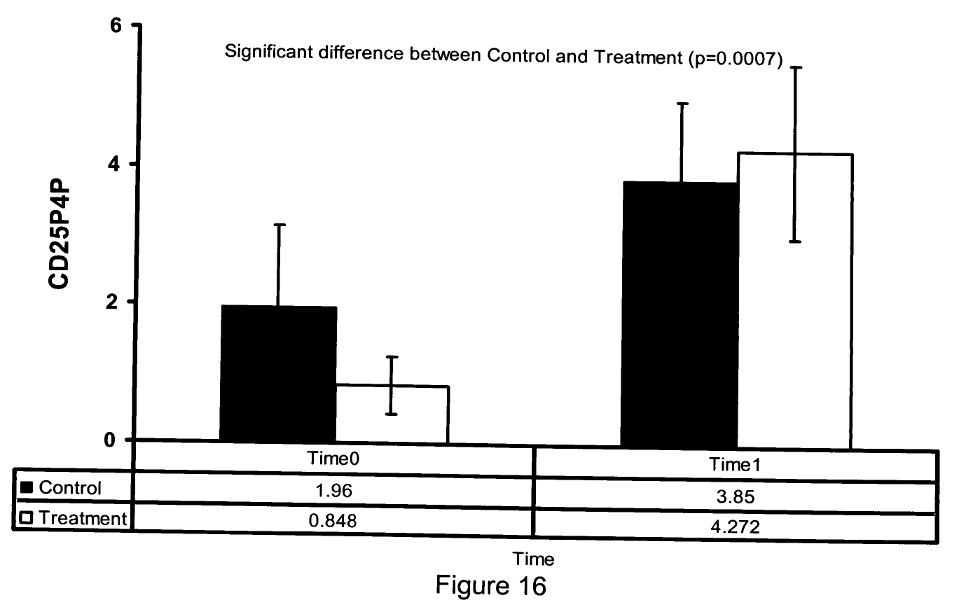

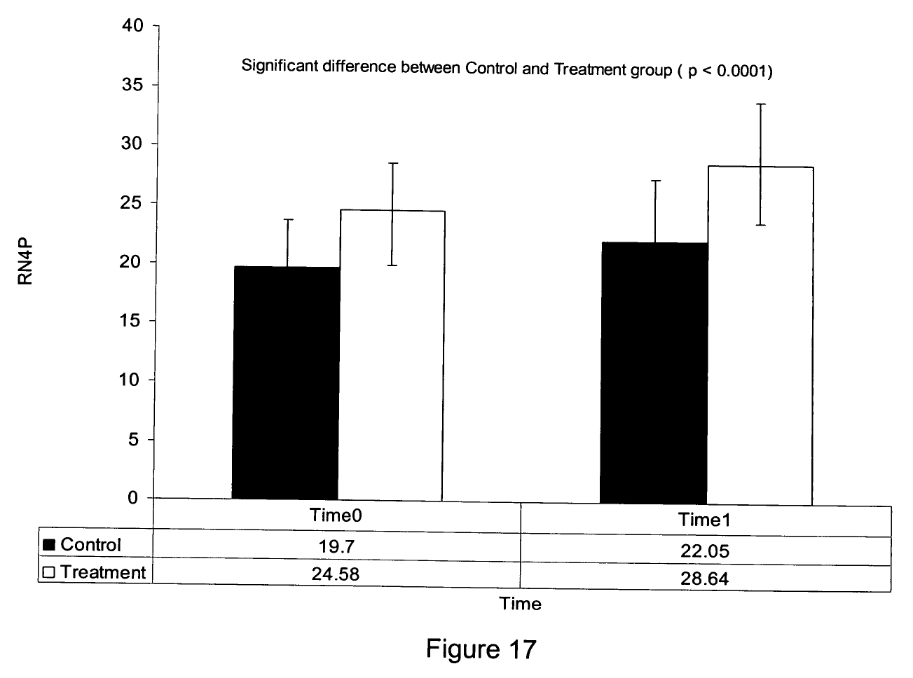

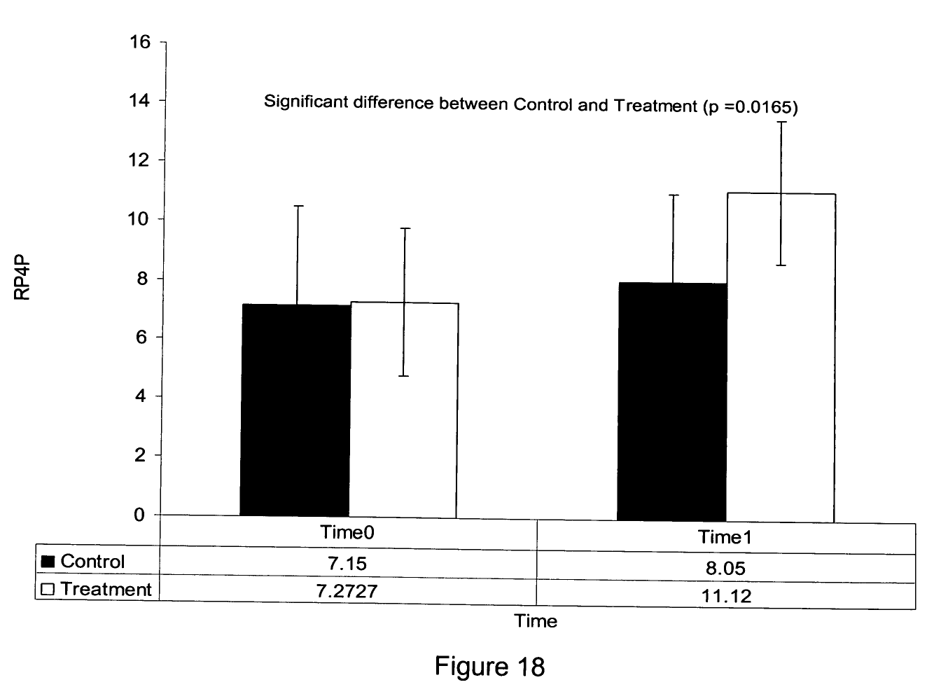

The same two groups of heifers described in Example 11 were further studied by assaying various immune markers (e.g. CD2, CD25+/CD4+, R−/4+ and R+/CD4+). Samples were assayed at Time 0 (prior to treatment), and Time 1 (18 days post-treatment). FIG. 15 shows the mean CD2 cell count in the treated and control groups pre- and post-treatment. FIG. 16 shows the mean CD25+/CD4+ cells in the treated and control groups pre- and post-treatment. FIG. 17 shows the mean R−/4+in the treated and control groups pre- and post-treatment. FIG. 18 shows the mean R+/CD4+ cells in the treated and control groups pre- and post-treatment. Treatment enhances the activated lymphocytes and natural killer cells.

Statistics. The data in the above examples were analyzed using Microsoft Excel statistics analysis package. Values shown in the figures are the mean±s.e.m. Specific p values will be obtained by comparison using Students t test. A p<0.05 was set as the level of statistical significance.

In contrast to injections with porcine recombinant somatotropin (rpST) or bST, which can produce unwanted side effects (e.g. hemorrhagic ulcers, vacuolations of liver and kidney or even death of the animals (Smith et al., 1991)), the plasmid mediated GHRH gene supplementation is well tolerated having no observed side effects in the animals. Regulated tissue/fiber-type-specific hGH-containing plasmids have been used previously for the delivery and stable production of GH in livestock and GH-deficient hosts. The methods used to deliver the hGH-containing plasmas comprise transgenesis, myoblast transfer or liposome-mediated intravenous injection (Barr and Leiden, 1991; Dahler et al., 1994; Pursel et al., 1990). Nevertheless, these techniques have significant disadvantages that preclude them from being used in a large-scale operation and/or on food animals, including: 1) possible toxicity or immune response associated with liposome delivery; 2) need for extensive ex vivo manipulation in the transfected myoblast approach; and/or 3) risk of important side effects or inefficiency in transgenesis (Dhawan et al., 1991; Miller et al., 1989). Compared to these techniques, plasmid mediated gene supplementation and DNA injection is simple and effective, with no complication related to the delivery system or to excess expression.

The embodiments provided herein illustrate that enhanced welfare of large mammals injected with a GHRH plasmid having decreased mortality and morbidity rates. Treated cows display a significantly higher milk production. Offspring calves did not experience any side effects from the therapy, including associated pathology or death. Although not wanting to be bound by theory, the profound enhancement in animal welfare indicates that ectopic expression of myogenic GHRH vectors will likely replace classical GH therapy regimens and may stimulate the GH axis in a more physiologically appropriate manner. The HV-GHRH molecule, which displays a high degree of stability and GH secretory activity in pigs, is also useful in other mammals, since the serum proteases that degrade GHRH are similar in most mammals.

One skilled in the art readily appreciates that this invention is well adapted to carry out the objectives and obtain the ends and advantages mentioned as well as those inherent therein. Growth hormone, growth hormone releasing hormone, analogs, plasmids, vectors, pharmaceutical compositions, treatments, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

### REFERENCES CITED

The entire content of each of the following U.S. patent documents and published references is hereby incorporated by reference.

### U.S. PATENT DOCUMENTS

U.S. Pat. No. 5,847,066 issued on Dec. 8, 1998 with Coy et al. listed as inventors.

U.S. Pat. No. 5,846,936 issued on Dec. 8, 1998 with Felix et al. listed as inventors.

U.S. Pat. No. 5,792,747 issued on Aug. 11, 1998 with Schally et al. listed as inventors.

U.S. Pat. No. 5,776,901 issued on Jul. 7, 1998 with Bowers et al. listed as inventors.

U.S. Pat. No. 5,756,264 issued on May 26, 1998 with Schwartz et al. listed as inventors.

U.S. Pat. No. 5,696,089 issued on Dec. 9, 1997 with Felix et al. listed as inventors.

U.S. Pat. No. 5,486,505 issued on Jan. 23, 1996 with Bowers et al. listed as inventors.

U.S. Pat. No. 5,292,721 issued on Mar. 8, 1994 with Boyd et al. listed as inventors.

U.S. Pat. No. 5,137,872 issued on Aug. 11, 1992 with Seely et al. listed as inventors.

U.S. Pat. No. 5,134,210 issued on Jul. 28, 1992 with Boyd et al. listed as inventors.

U.S. Pat. No. 5,084,442 issued on Jan. 28, 1992 with Felix et al. listed as inventors.

U.S. Pat. No. 5,061,690 issued on Oct. 29, 1991 with Kann et al. listed as inventors.

U.S. Pat. No. 5,036,045 issued on Jul. 30, 1991 with Thomer listed as the inventor.

U.S. Pat. No. 5,023,322 issued on Jun. 11, 1991 with Kovacs et al. listed as inventors.

U.S. Pat. No. 4,839,344 issued on Jun. 13, 1989 with Bowers et al. listed as inventors.

U.S. Pat. No. 4,410,512 issued on Oct. 18, 1983 with Bowers et al. listed as inventors.

U.S. Pat. No. RE33,699 issued on Sep. 24, 1991 with Drengler listed as the inventor.

U.S. Pat. No. 4,833,166 issued on May 23, 1989 with Grosvenor et al. listed as inventors.

U.S. Pat. No. 4,228,158 issued on Oct. 14, 1980 with Momany et al. listed as inventors.

U.S. Pat. No. 4,228,156 issued on Oct. 14, 1980 with Momany et al. listed as inventors.

U.S. Pat. No. 4,226,857 issued on Oct. 7, 1980 with Momany et al. listed as inventors.

U.S. Pat. No. 4,224,316 issued on Sep. 23, 1980 with Momany et al. listed as inventors.

U.S. Pat. No. 4,223,021 issued on Sep. 16, 1980 with Momany et al. listed as inventors.

U.S. Pat. No. 4,223,020 issued on Sep. 16, 1980 with Momany et al. listed as inventors.

U.S. Pat. No. 4,223,019 issued on Sep. 16, 1980 with Momany et al. listed as inventors.

### REFERENCE LIST

Aihara, H. and J. Miyazaki. 1998. Gene transfer into muscle by electroporation in vivo. Nat. Biotechnol. 16:867-870.

Almendro, N., T. Bellon, C. Rius, P. Lastres, C. Langa, A. Corbi, and C. Bemabeu. 1996. Cloning of the human platelet endothelial cell adhesion molecule-1 promoter and its tissue-specific expression. Structural and functional characterization. J. Immunol. 157:5411-5421.

Aratani, Y., R. Okazaki, and H. Koyama. 1992. End extension repair of introduced targeting vectors mediated by homologous recombination in mammalian cells. Nucleic Acids Res. 20:4795-4801.

Argente, J., J. Pozo, and J. A. Chowen. 1996. The growth hormone axis: control and effects. Hormone Research 45 Suppl 1:9-11.

Auchtung, T. L., D. S. Buchanan, C. A. Lents, S. M. Barao, and G. E. Dahl. 2001. Growth hormone response to growth hormone-releasing hormone in beef cows divergently selected for milk production. J. Anim Sci. 79:1295-1300.

Barr, E. and J. M. Leiden. 1991. Systemic delivery of recombinant proteins by genetically modified myoblasts [see comments]. Science 254:1507-1509.

Bercu, B. B. and R. F. Walker. 1997. Growth Hormone Secretagogues In Children With Altered Growth. Acta Paediatrica 86:102-106.

Bettan, M., F. Emmanuel, R. Darteil, J. M. Caillaud, F. Soubrier, P. Delaere, D. Branelec, A. Mahfoudi, N. Duverger, and D. Scherman. 2000. High-level protein secretion into blood circulation after electric pulse-mediated gene transfer into skeletal muscle. Mol. Ther. 2:204-210.

Blethen, S. L. and M. H. MacGillivray. 1997. A risk-benefit assessment of growth hormone use in children. Drug Saf 17:303-316.

Blethen, S. L. and A. C. Rundle. 1996. Slipped capital femoral epiphysis in children treated with growth hormone. A summary of the National Cooperative Growth Study experience. Horm. Res. 46:113-116.

Bohlen, P., F. Esch, P. Brazeau, N. Ling, and R. Guillemin. 1983. Isolation and characterization of the porcine hypothalamic growth hormone releasing factor. Biochem. Biophys. Res. Commun. 116:726-734.

Boshart, M., F. Weber, G. Jahn, K. Dorsch-Hasler, B. Fleckenstein, and W. Schaffner. 1985. A very strong enhancer is located upstream of an immediate early gene of human cytomegalovirus. Cell 41:521-530.

Carbonelli, D. L., E. Corley, M. Seigelchifer, and J. Zorzopulos. 1999. A plasmid vector for isolation of strong promoters in Escherichia coli. FEMS Microbiol. Lett. 177:75-82.

Chandler, S. D., A. Mayeda, J. M. Yeakley, A. R. Krainer, and X. D. Fu. 1997. RNA splicing specificity determined by the coordinated action of RNA recognition motifs in SR proteins. Proc. Natl. Acad. Sci. U.S. A 94:3596-3601.

Chevalier, R. L., S. Goyal, A. Kim, A. Y. Chang, D. Landau, and D. LeRoith. 2000. Renal tubulointerstitial injury from ureteral obstruction in the neonatal rat is attenuated by IGF-1. Kidney Int. 57:882-890.

Chung, C. S., T. D. Etherton, and J. P. Wiggins. 1985. Stimulation of swine growth by porcine growth hormone. J. Anim Sci. 60:118-130.

Cocea, L. 1997. Duplication of a region in the multiple cloning site of a plasmid vector to enhance cloning-mediated addition of restriction sites to a DNA fragment. Biotechniques 23:814-816.

Corpas, E., S. M. Harman, M. A. Pineyro, R. Roberson, and M. R. Blackman. 1993. Continuous subcutaneous infusions of growth hormone (GH) releasing hormone 1-44 for 14 days increase GH and insulin-like growth factor-I levels in old men. Journal of Clinical Endocrinology & Metabolism 76:134-138.

Dahl, G. E., L. T. Chapin, M. S. Allen, W. M. Moseley, and H. A. Tucker. 1991. Comparison of somatotropin and growth hormone-releasing factor on milk yield, serum hormones, and energy status. J. Dairy Sci. 74:3421-3428.

Dahler, A., R. P. Wade, G. E. Muscat, and M. J. Waters. 1994. Expression vectors encoding human growth hormone (hGH) controlled by human muscle-specific promoters: prospects for regulated production of hGH delivered by myoblast transfer or intravenous injection. Gene 145:305-310.

Dai, B., H. Wu, E. Holthuizen, and P. Singh. 2001. Identification of a novel cis element required for cell density-dependent down-regulation of insulin-like growth factor-2 P3 promoter activity in Caco2 cells. J. Biol. Chem. 276:6937-6944.

Danko, I. and J. A. Wolff. 1994. Direct gene transfer into muscle. [Review]. Vaccine 12:1499-1502.

Darquet, A. M., B. Cameron, P. Wils, D. Scherman, and J. Crouzet. 1997. A new DNA vehicle for nonviral gene delivery: supercoiled minicircle. Gene Ther. 4:1341-1349.

Darquet, A. M., R. Rangara, P. Kreiss, B. Schwartz, S. Naimi, P. Delaere, J. Crouzet, and D. Scherman. 1999. Minicircle: an improved DNA molecule for in vitro and in vivo gene transfer. Gene Ther. 6:209-218.

Davis, H. L., R. G. Whalen, and B. A. Demeneix. 1993. Direct gene transfer into skeletal muscle in vivo: factors affecting efficiency of transfer and stability of expression. Human Gene Therapy 4:151-159.

Dechow, C. D., G. W. Rogers, and J. S. Clay. 2002. Heritability and correlations among body condition score loss, body condition score, production and reproductive performance. J Dairy Sci 85:3062-3070.

Dhawan, J., L. C. Pan, G. K. Pavlath, M. A. Travis, A. M. Lanctot, and H. M. Blau. 1991. Systemic delivery of human growth hormone by injection of genetically engineered myoblasts. Science 254:1509-1512.

Dolnik, V., M. Novotny, and J. Chmelik. 1993. Electromigration behavior of poly-(L-glutamate) conformers in concentrated polyacrylamide gels. Biopolymers 33:1299-1306.

Domecq, J. J., A. L. Skidmore, J. W. Lloyd, and J. B. Kaneene. 1997. Relationship between body condition scores and milk yield in a large dairy herd of high yielding Holstein cows. J Dairy Sci 80:101-112.

Dorsch-Hasler, K., G. M. Keil, F. Weber, M. Jasin, W. Schaffner, and U. H. Koszinowski. 1985. A long and complex enhancer activates transcription of the gene coding for the highly abundant immediate early mRNA in murine cytomegalovirus. Proc. Natl. Acad. Sci. U.S. A 82:8325-8329.

Draghia-Akli, R., M. L. Fiorotto, L. A. Hill, P. B. Malone, D. R. Deaver, and R. J. Schwartz. 1999. Myogenic expression of an injectable protease-resistant growth hormone-releasing hormone augments long-term growth in pigs. Nat. Biotechnol. 17:1179-1183.

Draghia-Akli, R., A. S. Khan, K. K. Cummings, D. Parghi, R. H. Carpenter, and P. A. Brown. 2002a. Electrical Enhancement of Formulated Plasmid Delivery in Animals. Technology in Cancer Research & Treatment 1:365-371.

Draghia-Akli, R., X. G. Li, and R. J. Schwartz. 1997. Enhanced growth by ectopic expression of growth hormone releasing hormone using an injectable myogenic vector. Nat. Biotechnol. 15:1285-1289.

Draghia-Akli, R., P. B. Malone, L. A. Hill, K. M. Ellis, R. J. Schwartz, and J. L. Nordstrom. 2002b. Enhanced animal growth via ligand-regulated GHRH myogenic-injectable vectors. FASEB J. 16:426-428.

Dubreuil, P., D. Petitclerc, G. Pelletier, P. Gaudreau, C. Farmer, Mowles, TF, and P. Brazeau. 1990. Effect of dose and frequency of administration of a potent analog of human growth hormone-releasing factor on hormone secretion and growth in pigs. Journal of Animal Science 68:1254-1268.

Duck, S. C., H. P. Schwarz, G. Costin, R. Rapaport, S. Arslanian, A. Hayek, M. Connors, and J. Jaramillo. 1992. Subcutaneous growth hormone-releasing hormone therapy in growth hormone-deficient children: first year of therapy. Journal of Clinical Endocrinology & Metabolism 75:1115-1120.

Esch, F. S., P. Bohlen, N. C. Ling, P. E. Brazeau, W. B. Wehrenberg, M. O. Thorner, M. J. Cronin, and R. Guillemin. 1982. Characterization of a 40 residue peptide from a human pancreatic tumor with growth hormone releasing activity. Biochemical & Biophysical Research Communications 109:152-158.

Etherton, T. D., J. P. Wiggins, C. S. Chung, C. M. Evock, J. F. Rebhun, and P. E. Walton. 1986. Stimulation of pig growth performance by porcine growth hormone and growth hormone-releasing factor. Journal of Animal Science 63:1389-1399.

Etienne, M., M. Bonneau, G. Kann, and F. Deletang. 1992. Effects of administration of growth hormone-releasing factor to sows during late gestation on growth hormone secretion, reproductive traits, and performance of progeny from birth to 100 kilograms live weight. J Anim Sci 70:2212-2220.

Evans, W. S., M. L. Vance, D. L. Kaiser, R. P. Sellers, J. L. Borges, T. R. Downs, L. A. Frohman, J. Rivier, W. Vale, and M. O. Thorner. 1985. Effects of intravenous, subcutaneous, and intranasal administration of growth hormone (GH)-releasing hormone-40 on serum GH concentrations in normal men. Journal of Clinical Endocrinology & Metabolism 61:846-850.

Farmer, C., D. Petitclerc, G. Pelletier, and P. Brazeau. 1992. Lactation performance of sows injected with growth hormone-releasing factor during gestation and(or) lactation. Journal of Animal Science 70:2636-2642.

Farmer, C., S. Robert, and J. J. Matte. 1996. Lactation performance of sows fed a bulky diet during gestation and receiving growth hormone-releasing factor during lactation. J. Anim Sci. 74:1298-1306.

Fewell, J. G., F. MacLaughlin, V. Mehta, M. Gondo, F. Nicol, E. Wilson, and L. C. Smith. 2001. Gene therapy for the treatment of hemophilia B using PINC-formulated plasmid delivered to muscle with electroporation. Mol. Ther. 3:574-583.

Frohman, L. A., T. R. Downs, E. P. Heimer, and A. M. Felix. 1989. Dipeptidylpeptidase IV and trypsin-like enzymatic degradation of human growth hormone-releasing hormone in plasma. J. Clin. Invest. 83:1533-1540.

Frohman, L. A., J. L. Thominet, C. B. Webb, M. L. Vance, H. Uderman, J. Rivier, W. Vale, and M. O. Thomer. 1984. Metabolic clearance and plasma disappearance rates of human pancreatic tumor growth hormone releasing factor in man. J. Clin. Invest. 73:1304-1311.

Fryer, A. D. and D. B. Jacoby. 1993. Effect of inflammatory cell mediators on M2 muscarinic receptors in the lungs. Life Sci. 52:529-536.

Gehl, J., T. Skovsgaard, and L. M. Mir. 1998. Enhancement of cytotoxicity by electropermeabilization: an improved method for screening drugs. Anticancer Drugs 9:319-325.

Gehl, J., T. H. Sorensen, K. Nielsen, P. Raskmark, S. L. Nielsen, T. Skovsgaard, and L. M. Mir. 1999. In vivo electroporation of skeletal muscle: threshold, efficacy and relation to electric field distribution. Biochim. Biophys. Acta 1428:233-240.

German, M., S. Ashcroft, K. Docherty, H. Edlund, T. Edlund, S. Goodison, H. Imura, G. Kennedy, O. Madsen, D. Melloul, and. 1995. The insulin gene promoter. A simplified nomenclature. Diabetes 44:1002-1004.

Gopinath, R. and T. D. Etherton. 1989a. Effects of porcine growth hormone on glucose metabolism of pigs: I. Acute and chronic effects on plasma glucose and insulin status. J. Anim Sci. 67:682-688.

Gopinath, R. and T. D. Etherton. 1989b. Effects of porcine growth hormone on glucose metabolism of pigs: II. Glucose tolerance, peripheral tissue insulin sensitivity and glucose kinetics. J. Anim Sci. 67:689-697.

Guillemin, R., P. Brazeau, P. Bohlen, F. Esch, N. Ling, and W. B. Wehrenberg. 1982. Growth hormone-releasing factor from a human pancreatic tumor that caused acromegaly. Science 218:585-587.

Hammond, B. G., R. J. Collier, M. A. Miller, M. McGrath, D. L. Hartzell, C. Kotts, and W. Vandaele. 1990. Food safety and pharmacokinetic studies which support a zero (0) meat and milk withdrawal time for use of sometribove in dairy cows. Ann. Rech. Vet. 21 Suppl 1:107S-120S.:107S-120S.

Heller, R., M. J. Jaroszeski, L. F. Glass, J. L. Messina, D. P. Rapaport, R. C. DeConti, N. A. Fenske, R. A. Gilbert, L. M. Mir, and D. S. Reintgen. 1996. Phase I/II trial for the treatment of cutaneous and subcutaneous tumors using electrochemotherapy. Cancer 77:964-971.

Herzog, R. W., J. D. Mount, V. R. Arruda, K. A. High, and C. D. Lothrop, Jr. 2001. Muscle-directed gene transfer and transient immune suppression result in sustained partial correction of canine hemophilia B caused by a null mutation. Mol. Ther. 4:192-200.

Horlick, R. A. and P. A. Benfield. 1989. The upstream muscle-specific enhancer of the rat muscle creatine kinase gene is composed of multiple elements. Mol. Cell Biol. 9:2396-2413.

Inouye, C., P. Remondelli, M. Karin, and S. Elledge. 1994. Isolation of a cDNA encoding a metal response element binding protein using a novel expression cloning procedure: the one hybrid system. DNA Cell Biol. 13:731-742.

Inouye, S., A. Nakazawa, and T. Nakazawa. 1985. Determination of the transcription initiation site and identification of the protein product of the regulatory gene xylR for xyl operons on the TOL plasmid. J. Bacteriol. 163:863-869.

Jardieu, P., R. Clark, D. Mortensen, and K. Dorshkind. 1994. In vivo administration of insulin-like growth factor-I stimulates primary B lymphopoiesis and enhances lymphocyte recovery after bone marrow transplantation. J. Immunol. 152:4320-4327.

Jaynes, J. B., J. E. Johnson, J. N. Buskin, C. L. Gartside, and S. D. Hauschka. 1988. The muscle creatine kinase gene is regulated by multiple upstream elements, including a muscle-specific enhancer. Mol. Cell Biol. 8:62-70.

Kawamoto, T., K. Makino, H. Niwa, H. Sugiyama, S. Kimura, M. Amemura, A. Nakata, and T. Kakunaga. 1988. Identification of the human beta-actin enhancer and its binding factor. Mol. Cell Biol. 8:267-272.

Kawamoto, T., K. Makino, S. Orita, A. Nakata, and T. Kakunaga. 1989. DNA bending and binding factors of the human beta-actin promoter. Nucleic Acids Res. 17:523-537.

Klamut, H. J., L. O. Bosnoyan-Collins, R. G. Worton, P. N. Ray, and H. L. Davis. 1996. Identification of a transcriptional enhancer within muscle intron 1 of the human dystrophin gene. Hum. Mol. Genet. 5:1599-1606.

Klamut, H. J., S. B. Gangopadhyay, R. G. Worton, and P. N. Ray. 1990. Molecular and functional analysis of the muscle-specific promoter region of the Duchenne muscular dystrophy gene. Mol. Cell Biol. 10:193-205.

Klindt, J., J. T. Yen, F. C. Buonomo, A. J. Roberts, and T. Wise. 1998. Growth, body composition, and endocrine responses to chronic administration of insulin-like growth factor I and(or) porcine growth hormone in pigs. J. Anim Sci. 76:2368-2381.

Kraus, J., M. Woltje, N. Schonwetter, and V. Hollt. 1998. Alternative promoter usage and tissue specific expression of the mouse somatostatin receptor 2 gene. FEBS Lett. 428:165-170.

Lareyre, J. J., T. Z. Thomas, W. L. Zheng, S. Kasper, D. E. Ong, M. C. Orgebin-Crist, and R. J. Matusik. 1999. A 5-kilobase pair promoter fragment of the murine epididymal retinoic acid-binding protein gene drives the tissue-specific, cell-specific, and androgen-regulated expression of a foreign gene in the epididymis of transgenic mice. J. Biol. Chem. 274:8282-8290.

Larsen, P. R., J. W. Harney, and D. D. Moore. 1986. Sequences required for cell-type specific thyroid hormone regulation of rat growth hormone promoter activity. J. Biol. Chem. 261:14373-14376.

Lee, S. H., W. Wang, S. Yajima, P. A. Jose, and M. M. Mouradian. 1997. Tissue-specific promoter usage in the DIA dopamine receptor gene in brain and kidney. DNA Cell Biol. 16:1267-1275.

Lesbordes, J. C., T. Bordet, G. Haase, L. Castelnau-Ptakhine, S. Rouhani, H. Gilgenkrantz, and A. Kahn. 2002. In vivo electrotransfer of the cardiotrophin-1 gene into skeletal muscle slows down progression of motor neuron degeneration in pmn mice. Hum. Mol. Genet. 11:1615-1625.

Levenson, V. V., E. D. Transue, and I. B. Roninson. 1998. Internal ribosomal entry site-containing retroviral vectors with green fluorescent protein and drug resistance markers. Hum. Gene Ther. 9:1233-1236.

Li, C., S. Ke, Q. P. Wu, W. Tansey, N. Hunter, L. M. Buchmiller, L. Milas, C. Charnsangavej, and S. Wallace. 2000. Tumor irradiation enhances the tumor-specific distribution of poly(L-glutamic acid)-conjugated paclitaxel and its antitumor efficacy. Clin. Cancer Res. 6:2829-2834.

Li, X., E. M. Eastman, R. J. Schwartz, and R. Draghia-Akli. 1999. Synthetic muscle promoters: activities exceeding naturally occurring regulatory sequences. Nat. Biotechnol. 17:241-245.

Lin, H., K. E. Yutzey, and S. F. Konieczny. 1991. Muscle-specific expression of the troponin I gene requires interactions between helix-loop-helix muscle regulatory factors and ubiquitous transcription factors. Mol. Cell Biol. 11:267-280.

Liu, Y., H. Li, K. Tanaka, N. Tsumaki, and Y. Yamada. 2000. Identification of an enhancer sequence within the first intron required for cartilage-specific transcription of the alpha2(XI) collagen gene. J. Biol. Chem. 275:12712-12718.

Lucas, M. L., L. Heller, D. Coppola, and R. Heller. 2002. IL-12 plasmid delivery by in vivo electroporation for the successful treatment of established subcutaneous B16.F10 melanoma. Mol. Ther. 5:668-675.

Lucas, M. L., M. J. Jaroszeski, R. Gilbert, and R. Heller. 2001. In vivo electroporation using an exponentially enhanced pulse: a new waveform. DNA Cell Biol. 20:183-188.

Macejak, D. G. and P. Sarnow. 1991. Internal initiation of translation mediated by the 5′ leader of a cellular mRNA. Nature 353:90-94.

Matsubara, H., Y. Gunji, T. Maeda, K. Tasaki, Y. Koide, T. Asano, T. Ochiai, S. Sakiyama, and M. Tagawa. 2001. Electroporation-mediated transfer of cytokine genes into human esophageal tumors produces anti-tumor effects in mice. Anticancer Res. 21:2501-2503.

Matsuo, A., I. Tooyama, S. Isobe, Y. Oomura, I. Akiguchi, K. Hanai, J. Kimura, and H. Kimura. 1994. Immunohistochemical localization in the rat brain of an epitope corresponding to the fibroblast growth factor receptor-1. Neuroscience 60:49-66.

McNally, M. A., J. S. Lebkowski, T. B. Okarma, and L. B. Lerch. 1988. Optimizing electroporation parameters for a variety of human hematopoietic cell lines. Biotechniques 6:882-886.

Miklavcic, D., K. Beravs, D. Semrov, M. Cemazar, F. Demsar, and G. Sersa. 1998. The importance of electric field distribution for effective in vivo electroporation of tissues. Biophys. J 74:2152-2158.

Miller, K. F., D. J. Bolt, V. G. Pursel, R. E. Hammer, C. A. Pinkert, R. D. Palmiter, and R. L. Brinster. 1989. Expression of human or bovine growth hormone gene with a mouse metallothionein-1 promoter in transgenic swine alters the secretion of porcine growth hormone and insulin-like growth factor-I. J. Endocrinol. 120:481-488.

Mumper, R. J., J. Wang, S. L. Klakamp, H. Nitta, K. Anwer, F. Tagliaferri, and A. P. Rolland. 1998. Protective interactive noncondensing (PINC) polymers for enhanced plasmid distribution and expression in rat skeletal muscle. J. Control Release 52:191-203.

Muramatsu, T., S. Arakawa, K. Fukazawa, Y. Fujiwara, T. Yoshida, R. Sasaki, S. Masuda, and H. M. Park. 2001. In vivo gene electroporation in skeletal muscle with special reference to the duration of gene expression. Int. J. Mol. Med. 7:37-42.

Murray, R. D. and S. M. Shalet. 2000. Growth hormone: current and future therapeutic applications. Expert. Opin. Pharmacother. 1:975-990.

Nairn, R. S., G. M. Adair, T. Porter, S. L. Pennington, D. G. Smith, J. H. Wilson, and M. M. Seidman. 1993. Targeting vector configuration and method of gene transfer influence targeted correction of the APRT gene in Chinese hamster ovary cells. Somat. Cell Mol. Genet. 19:363-375.

Narum, D. L., S. Kumar, W. O. Rogers, S. R. Fuhrmann, H. Liang, M. Oakley, A. Taye, B. K. Sim, and S. L. Hoffman. 2001. Codon optimization of gene fragments encoding Plasmodium falciparum merzoite proteins enhances DNA vaccine protein expression and immunogenicity in mice. Infect. Immun. 69:7250-7253.

Neumann, E., M. Schaefer-Ridder, Y. Wang, and P. H. Hofschneider. 1982. Gene transfer into mouse lyoma cells by electroporation in high electric fields. EMBO J. 1:841-845.

Nomoto, S., Y. Tatematsu, T. Takahashi, and H. Osada. 1999. Cloning and characterization of the alternative promoter regions of the human LIMK2 gene responsible for alternative transcripts with tissue-specific expression. Gene 236:259-271.

Ohlsson, H., S. Thor, and T. Edlund. 1991. Novel insulin promoter- and enhancer-binding proteins that discriminate between pancreatic alpha- and beta-cells. Mol. Endocrinol. 5:897-904.

Otani, Y., Y. Tabata, and Y. Ikada. 1996. Rapidly curable biological glue composed of gelatin and poly(L-glutamic acid). Biomaterials 17:1387-1391.

Otani, Y., Y. Tabata, and Y. Ikada. 1998. Hemostatic capability of rapidly curable glues from gelatin, poly(L-glutamic acid), and carbodiimide. Biomaterials 19:2091-2098.

Parker, R. Using Body Condition Scoring in Dairy Herd Management. Ontario Ministry of Agriculture and Food Fact Sheet # 94-053. 1996.

Pech, M., C. D. Rao, K. C. Robbins, and S. A. Aaronson. 1989. Functional identification of regulatory elements within the promoter region of platelet-derived growth factor 2. Mol. Cell Biol. 9:396-405.

Pelletier, J. and N. Sonenberg. 1988. Internal initiation of translation of eukaryotic mRNA directed by a sequence derived from poliovirus RNA. Nature 334:320-325.

Pinkert, C. A., D. M. Omitz, R. L. Brinster, and R. D. Palmiter. 1987. An albumin enhancer located 10 kb upstream functions along with its promoter to direct efficient, liver-specific expression in transgenic mice. Genes Dev. 1:268-276.

Potter, H., L. Weir, and P. Leder. 1984. Enhancer-dependent expression of human kappa immunoglobulin genes introduced into mouse pre-B lymphocytes by electroporation. Proc. Natl. Acad. Sci. U.S. A 81:7161-7165.

Prentice, H., R. A. Kloner, T. Prigozy, T. Christensen, L. Newman, Y. Li, and L. Kedes. 1994. Tissue restricted gene expression assayed by direct DNA injection into cardiac and skeletal muscle. Journal of Molecular & Cellular Cardiology 26:1393-1401.

Pursel, V. G., D. J. Bolt, K. F. Miller, C. A. Pinkert, R. E. Hammer, R. D. Palmiter, and R. L. Brinster. 1990. Expression and performance in transgenic pigs. J. Reprod. Fertil. Suppl 40:235-45:235-245.

Radke, B. and G. Shook. 2001. Culling and Genetic Improvement Programs For Dairy Herds. In: Food Animal Production Medicine. pp. 291-307. W.B.Saunders Company.

Robbins, K., S. McCabe, T. Scheiner, J. Strasser, R. Clark, and P. Jardieu. 1994. Immunological effects of insulin-like growth factor-I—enhancement of immunoglobulin synthesis. Clin. Exp. Immunol. 95:337-342.

Rodenburg, J. Body Condition Scoring of Cattle. Ontario Ministry of Agriculture and Food Fact Sheet # 92-122. 1996.

Satozawa, N., K. Takezawa, T. Miwa, S. Takahashi, M. Hayakawa, and H. Ooka. 2000. Differences in the effects of 20 K- and 22 K-hGH on water retention in rats. Growth Horm. IGF. Res. 10: 187-192.

Skroch, P., C. Buchman, and M. Karin. 1993. Regulation of human and yeast metallothionein gene transcription by heavy metal ions. Prog. Clin. Biol. Res. 380:113-28.:113-128.

Smith, L. C. and J. L. Nordstrom. 2000. Advances in plasmid gene delivery and expression in skeletal muscle. Curr. Opin. Mol. Ther. 2:150-154.

Smith, V. G., A. D. Leman, W. J. Seaman, and F. VanRavenswaay. 1991. Pig weaning weight and changes in hematology and blood chemistry of sows injected with recombinant porcine somatotropin during lactation. J. Anim Sci. 69:3501-3510.

Song, S., J. Embury, P. J. Laipis, K. I. Bems, J. M. Crawford, and T. R. Flotte. 2001. Stable therapeutic serum levels of human alpha-1 antitrypsin (AAT) after portal vein injection of recombinant adeno-associated virus (rAAV) vectors. Gene Ther. 8:1299-1306.

Soubrier, F., B. Cameron, B. Manse, S. Somarriba, C. Dubertret, G. Jaslin, G. Jung, C. L. Caer, D. Dang, J. M. Mouvault, D. Scherman, J. F. Mayaux, and J. Crouzet. 1999. pCOR: a new design of plasmid vectors for nonviral gene therapy. Gene Ther. 6:1482-1488.

Studer, E. 1998. A veterinary perspective of on-farm evaluation of nutrition and reproduction. J Dairy Sci 81:872-876.

Terada, Y., H. Tanaka, T. Okado, S. Inoshita, M. Kuwahara, T. Akiba, S. Sasaki, and F. Marumo. 2001. Efficient and ligand-dependent regulated erythropoietin production by naked dna injection and in vivo electroporation. Am. J. Kidney Dis. 38:S50-S53.

Thorner, M. O., L. A. Frohman, D. A. Leong, J. Thominet, T. Downs, P. Hellmann, J. Chitwood, J. M. Vaughan, and W. Vale. 1984. Extrahypothalamic growth-hormone-releasing factor (GRF) secretion is a rare cause of acromegaly: plasma GRF levels in 177 acromegalic patients. Journal of Clinical Endocrinology & Metabolism 59:846-849.

Thorner, M. O., M. L. Hartman, M. L. Vance, S. S. Pezzoli, and E. J. Ampleford. 1995. Neuroendocrine regulation of growth hormone secretion. Neuroscience & Biobehavioral Reviews 19:465-468.

Thorner, M. O., M. L. Vance, W. S. Evans, A. D. Rogol, J. Rivier, W. Vale, Blizzard, and RM. 1986. Clinical studies with GHRH in man. Hormone Research 24:91-98.

Toneguzzo, F., A. Keating, S. Glynn, and K. McDonald. 1988. Electric field-mediated gene transfer: characterization of DNA transfer and patterns of integration in lymphoid cells. Nucleic Acids Res. 16:5515-5532.

Tripathy, S. K., E. C. Svensson, H. B. Black, E. Goldwasser, M. Margalith, Hobart, P M, and J. M. Leiden. 1996. Long-term expression of erythropoietin in the systemic circulation of mice after intramuscular injection of a plasmid DNA vector. Proc. Natl. Acad. Sci. USA 93:10876-10880.

Tronche, F., A. Rollier, I. Bach, M. C. Weiss, and M. Yaniv. 1989. The rat albumin promoter: cooperation with upstream elements is required when binding of APF/HNF1 to the proximal element is partially impaired by mutation or bacterial methylation. Mol. Cell Biol. 9:4759-4766.

Tronche, F., A. Rollier, P. Herbomel, I. Bach, S. Cereghini, M. Weiss, and M. Yaniv. 1990. Anatomy of the rat albumin promoter. Mol. Biol. Med. 7:173-185.

Trudel, M. and F. Costantini. 1987. A 3′ enhancer contributes to the stage-specific expression of the human beta-globin gene. Genes Dev. 1:954-961.

Tsumaki, N., T. Kimura, K. Tanaka, J. H. Kimura, T. Ochi, and Y. Yamada. 1998. Modular arrangement of cartilage- and neural tissue-specific cis-elements in the mouse alpha2(XI) collagen promoter. J. Biol. Chem. 273:22861-22864.

Tsunekawa, B., M. Wada, M. Ikeda, H. Uchida, N. Naito, and M. Honjo. 1999. The 20-kilodalton (kDa) human growth hormone (hGH) differs from the 22-kDa hGH in the effect on the human prolactin receptor. Endocrinology 140:3909-3918.

Tsurumi, Y., S. Takeshita, D. Chen, M. Kearney, S. T. Rossow, J. Passeri, J. R. Horowitz, J. F. Symes, and J. M. Isner. 1996. Direct intramuscular gene transfer of naked DNA encoding vascular endothelial growth factor augments collateral development and tissue perfusion [see comments]. Circulation 94:3281-3290.

Tur-Kaspa, R., L. Teicher, B. J. Levine, A. I. Skoultchi, and D. A. Shafritz. 1986. Use of electroporation to introduce biologically active foreign genes into primary rat hepatocytes. Mol. Cell Biol. 6:716-718.

Vance, M. L. 1990. Growth-hormone-releasing hormone. [Review] [52 refs]. Clinical Chemistry 36:415-420.

Vance, M. L., D. L. Kaiser, W. S. Evans, R. Furlanetto, W. Vale, J. Rivier, and M. O. Thomer. 1985. Pulsatile growth hormone secretion in normal man during a continuous 24-hour infusion of human growth hormone releasing factor (1-40). Evidence for intermittent somatostatin secretion. J. Clin. Invest. 75:1584-1590.

Verhelst, J., R. Abs, M. Vandeweghe, J. Mockel, J. J. Legros, G. Copinschi, C. Mahler, B. Velkeniers, L. Vanhaelst, A. Van Aelst, D. De Rijdt, A. Stevenaert, and A. Beckers. 1997. Two years of replacement therapy in adults with growth hormone deficiency. Clin. Endocrinol. (Oxf) 47:485-494.

Vilquin, J. T., P. F. Kennel, M. Patumeau-Jouas, P. Chapdelaine, N. Boissel, P. Delaere, J. P. Tremblay, D. Scherman, M. Y. Fiszman, and K. Schwartz. 2001. Electrotransfer of naked DNA in the skeletal muscles of animal models of muscular dystrophies. Gene Ther. 8:1097-1107.

Vittone, J., M. R. Blackman, J. Busby-Whitehead, C. Tsiao, K. J. Stewart, J. Tobin, T. Stevens, M. F. Bellantoni, M. A. Rogers, G. Baumann, J. Roth, S. M. Harman, and R. G. S. Spencer. 1997. Effects of single nightly injections of growth hormone-releasing hormone (GHRH 1-29) in healthy elderly men. Metabolism: Clinical and Experimental 46:89-96.

Wada, M., H. Uchida, M. Ikeda, B. Tsunekawa, N. Naito, S. Banba, E. Tanaka, Y. Hashimoto, and M. Honjo. 1998. The 20-kilodalton (kDa) human growth hormone (hGH) differs from the 22-kDa hGH in the complex formation with cell surface hGH receptor and hGH-binding protein circulating in human plasma. Mol. Endocrinol. 12:146-156.

Wells, K. E., J. Maule, R. Kingston, K. Foster, J. McMahon, E. Damien, A. Poole, and D. J. Wells. 1997. Immune responses, not promoter inactivation, are responsible for decreased long-term expression following plasmid gene transfer into skeletal muscle. FEBS Lett. 407:164-168.

Wolff, J. A., R. W. Malone, P. Williams, W. Chong, G. Acsadi, A. Jani, Felgner, and PL. 1990. Direct gene transfer into mouse muscle in vivo. Science 247:1465-1468.

Wu, H. K., J. A. Squire, Q. Song, and R. Weksberg. 1997. Promoter-dependent tissue-specific expressive nature of imprinting gene, insulin-like growth factor II, in human tissues. Biochem. Biophys. Res. Commun. 233:221-226.

Xie, T. D. and T. Y. Tsong. 1993. Study of mechanisms of electric field-induced DNA transfection. V. Effects of DNA topology on surface binding, cell uptake, expression, and integration into host chromosomes of DNA in the mammalian cell. Biophys. J. 65:1684-1689.

Yasui, A., K. Oda, H. Usunomiya, K. Kakudo, T. Suzuki, T. Yoshida, H. M. Park, K. Fukazawa, and T. Muramatsu. 2001. Elevated gastrin secretion by in vivo gene electroporation in skeletal muscle. Int. J. Mol. Med. 8:489-494.

Yin, D. and J. G. Tang. 2001. Gene therapy for streptozotocin-induced diabetic mice by electroporational transfer of naked human insulin precursor DNA into skeletal muscle in vivo. FEBS Lett. 495:16-20.

Yorifuji, T. and H. Mikawa. 1990. Co-transfer of restriction endonucleases and plasmid DNA into mammalian cells by electroporation: effects on stable transformation. Mutat. Res. 243:121-126.

Yutzey, K. E. and S. F. Konieczny. 1992. Different E-box regulatory sequences are functionally distinct when placed within the context of the troponin I enhancer. Nucleic Acids Res. 20:5105-5113.

Zhao-Emonet, J. C., O. Boyer, J. L. Cohen, and D. Klatzmann. 1998. Deletional and mutational analyses of the human CD4 gene promoter: characterization of a minimal tissue-specific promoter. Biochim. Biophys. Acta 1442:109-119.

What is claimed is:

1. A method of decreasing an involuntary cull in farm animals comprising: delivering into a tissue of the farm animals an isolated nucleic acid expression construct that encodes a growth-hormone-releasing-hormone (“GHRH”) or functional biological equivalent thereof; wherein the involuntary cull comprises infection, disease, morbidity, or mortality of the farm animals.

2. The method of claim 1, wherein the involuntary cull from mortality is decreased from about 20% in farm animals not having the isolated nucleic acid expression construct delivered into a tissue to less than 15% in farm animals having the isolated nucleic acid expression construct delivered.

3. The method of claim 1, wherein the involuntary cull comprises mortality at birth of newborns of the farm animals.

4. The method of claim 1, wherein the involuntary cull comprises post-natal mortality of newborns of the farm animals.

5. The method of claim 1, wherein delivering into the tissue of the farm animals the isolated nucleic acid expression construct is via electroporation method, a viral vector, in conjunction with a carrier, by parenteral route, or a combination thereof.

6. The method of claim 5, wherein the electroporation method comprising:
(a) penetrating the tissue in the farm animals with a plurality of needle electrodes, wherein the plurality of needle electrodes are arranged in a spaced relationship;
(b) introducing the isolated nucleic acid expression construct into the tissue between the plurality of needle electrodes; and
(c) applying an electrical pulse to the plurality of needle electrodes.

7. The method of claim 1, wherein the isolated nucleic acid expression construct is delivered in a single dose.

8. The method of claim 7, wherein the single dose comprises about a 2 mg quantity of nucleic acid expression construct.

9. The method of claim 1, wherein the tissue of the farm animals comprise diploid cells.

10. The method of claim 1, wherein the tissue of the farm animals comprise muscle cells.

11. The method of claim 1, wherein the isolated nucleic acid expression construct comprises a HV-GHRH plasmid (SEQID#11).

12. The method of claim 1, wherein the isolated nucleic acid expression construct comprises an optimized pAV0204 bGHRH plasmid (SEQ ID#19).

13. The method of claim 1, wherein the isolated nucleic acid expression construct is a TI-GHRH plasmid (SEQ ID#12), TV-GHRH Plasmid (SEQ ID#13), 15/27/28 GHRH plasmid (SEQ ID#14), or pSP-wt-GHRH plasmid.

14. The method of claim 1, wherein the isolated nucleic acid expression construct is an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

15. The method of claim 1, wherein the isolated nucleic acid expression construct further comprises, a transfection-facilitating polypeptide.

16. The method of claim 15, wherein the transfection-facilitating polypeptide comprises a charged polypeptide.

17. The method of claim 15, wherein the transfection-facilitating polypeptide comprises poly-L-glutamate.

18. The method of claim 1, wherein the delivering into the cells of the farm animals the isolated nucleic acid expression construct initiates expression of the encoded GHRH or functional biological equivalent thereof.

19. The method of claim 1, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

20. The method of claim 1, wherein the encoded GHRH or functional biological equivalent thereof is of formula (SEQ ID No: 6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

21. The method of claim 1, wherein the farm animals comprises ruminant animals, food animals, or work animals.

22. The method of claim 1, wherein the farm animals comprise dairy cows.

23. A method of improving a body condition score (“BCS”) in farm animals comprising: delivering into a tissue of the farm animals an isolated nucleic acid expression construct that encodes a growth-hormone-releasing-hormone (“GHRH”) or functional biological equivalent thereof; wherein the BSC is an aid used to evaluate an overall nutritional state of the farm animals.

24. The method of claim 23, wherein delivering into the tissue of the farm animals the isolated nucleic acid expression construct is via electroporation method, a viral vector, in conjunction with a carrier, by parenteral route, or a combination thereof.

25. The method of claim 24, wherein the electroporation method comprising:
(a) penetrating the tissue in the farm animals with a plurality of needle electrodes, wherein the plurality of needle electrodes are arranged in a spaced relationship;
(b) introducing the isolated nucleic acid expression construct into the tissue between the plurality of needle electrodes; and
(c) applying an electrical pulse to the plurality of needle electrodes.

26. The method of claim 23, wherein the isolated nucleic acid expression construct is delivered in a single dose.

27. The method of claim 26, wherein the single dose comprises about a 2 mg quantity of nucleic acid expression construct.

28. The method of claim 26, wherein the tissues of the farm animals comprise diploid cells.

29. The method of claim 26, wherein the tissues of the farm animals comprise muscle cells.

30. The method of claim 26, wherein the isolated nucleic acid expression construct comprises a HV-GHRH plasmid (SEQ ID#11).

31. The method of claim 26, wherein the isolated nucleic acid expression construct comprises an optimized pAV0204 bGHRH plasmid (SEQ ID#19).

32. The method of claim 26, wherein the isolated nucleic acid expression construct is a TI-GHRH plasmid (SEQ ID#12), TV-GHRH Plasmid (SEQ ID#13), 15/27/28 GHRH plasmid (SEQ ID#14), or pSP-wt-GHRH plasmid.

33. The method of claim 26, wherein the isolated nucleic acid expression construct is an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

34. The method of claim 26, wherein the isolated nucleic acid expression construct further comprises, a transfection-facilitating polypeptide.

35. The method of claim 34, wherein the transfection-facilitating polypeptide comprises a charged polypeptide.

36. The method of claim 34, wherein the transfection-facilitating polypeptide comprises poly-L-glutamate.

37. The method of claim 26, wherein the delivering into the cells of the farm animals the isolated nucleic acid expression construct initiates expression of the encoded GHRH or functional biological equivalent thereof.

38. The method of claim 26, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

39. The method of claim 26, wherein the encoded GHRH or functional biological equivalent thereof is of formula (SEQ ID No: 6):
—X₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

40. The method of claim 26, wherein the farm animals comprises a ruminant animals, a food animals, or a work animals.

41. The method of claim 26, wherein the farm animals comprises a pig, sheep, goat or chicken.

42. The method of claim 26, wherein the farm animals comprise bovine.

43. The method of claim 26, wherein the farm animals comprise dairy cows.

44. A method of increasing milk production in a dairy cow comprising: delivering into muscle tissues of the dairy cow an isolated nucleic acid expression construct that encodes a growth-hormone-releasing-hormone (“GHRH”) or functional biological equivalent thereof;
wherein
delivering into the tissue of the farm animals the isolated nucleic acid expression construct is via electroporation, a viral vector, in conjunction with a carrier, by parenteral route, or a combination thereof; and
the isolated nucleic acid expression construct is delivered in a single dose.

45. The method of claim 44, wherein the increase in milk production is increased from about 8% to about 18% in farm animals having the isolated nucleic acid expression construct delivered when compared to animals not having the isolated nucleic acid expression construct delivered.

46. The method of claim 44, wherein the electroporation method comprising:
(a) penetrating the tissue in the farm animals with a plurality of needle electrodes, wherein the plurality of needle electrodes are arranged in a spaced relationship;
(b) introducing the isolated nucleic acid expression construct into the tissue between the plurality of needle electrodes; and
(c) applying an electrical pulse to the plurality of needle electrodes.

47. The method of claim 44, wherein the single dose comprises about a 2 mg quantity of nucleic acid expression construct.

48. The method of claim 44, wherein the isolated nucleic acid expression construct comprises a HV-GHRH plasmid (SEQ ID#11).

49. The method of claim 44, wherein the isolated nucleic acid expression construct comprises an optimized pAV0204 bGHRH plasmid (SEQ ID#19).

50. The method of claim 44, wherein the isolated nucleic acid expression construct is a TI-GHRH plasmid (SEQ ID#12), TV-GHRH Plasmid (SEQ ID#13), 15/27/28 GHRH plasmid (SEQ ID#14), or pSP-wt-GHRH plasmid.

51. The method of claim 44, wherein the isolated nucleic acid expression construct is an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

52. The method of claim 44, wherein the isolated nucleic acid expression construct further comprises, a transfection-facilitating polypeptide.

53. The method of claim 52, wherein the transfection-facilitating polypeptide comprises a charged polypeptide.

54. The method of claim 52, wherein the transfection-facilitating polypeptide comprises poly-L-glutamate.

55. The method of claim 44, wherein the delivering into the cells of the farm animals the isolated nucleic acid expression construct initiates expression of the encoded GHRH or functional biological equivalent thereof.

56. The method of claim 44, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

57. The method of claim 44, wherein the encoded GHRH or functional biological equivalent thereof is of formula (SEQ ID No: 6):
—X₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

58. A method of decreasing an involuntary cull in farm animals comprising: delivering into a muscle tissue of the farm animals an isolated nucleic acid expression construct that encodes a growth-hormone-releasing-hormone (“GHRH”) or functional biological equivalent thereof; wherein;
the involuntary cull comprises infection, disease, morbidity, or mortality of the farm animals;
delivering is via an in vivo electroporation method;
the isolated nucleic acid expression construct is delivered in a single dose; and
the encoded GHRH or functional biological equivalent thereof is of formula (SEQ ID No: 6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

59. The method of claim 58, wherein the involuntary cull comprises mortality at birth of newborns of the farm animals.

60. The method of claim 58, wherein the involuntary cull further comprises post-natal mortality of newborns of the farm animals.

61. The method of claim 58, wherein the single dose comprises about a 2 mg quantity of nucleic acid expression construct.

62. The method of claim 58, wherein the isolated nucleic acid expression construct is a HV-GHRH plasmid (SEQ ID#11), or an optimized pAV0204 bGHRH plasmid (SEQ ID#19).

63. The method of claim 58, wherein the isolated nucleic acid expression construct is a TI-GHRH plasmid (SEQ ID#12), TV-GHRH Plasmid (SEQ ID#13), 15/27/28 GHRH plasmid (SEQ ID#14), pSP-wt-GHRH plasmid, pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

64. The method of claim 58, wherein the isolated nucleic acid expression construct further comprises, poly-L-glutamate.

65. The method of claim 58, wherein the farm animals comprises a bovine.

66. A method of improving a body condition score (“BCS”) in farm animals comprising: delivering into a muscle tissue of the farm animals an isolated nucleic acid expression construct that encodes a growth-hormone-releasing-hormone (“GHRH”) or functional biological equivalent thereof;
wherein:
the BSC is an aid used to evaluate an overall nutritional state of the farm animals;
delivering is via an in vivo electroporation method;
the isolated nucleic acid expression construct is delivered in a single dose; and
the encoded GHRH or functional biological equivalent thereof is of formula (SEQ ID No: 6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

67. The method of claim 66, wherein the single dose comprises about a 2 mg quantity of nucleic acid expression construct.

68. The method of claim 66, wherein the isolated nucleic acid expression construct is a HV-GHRH plasmid (SEQ ID#11), or an optimized pAV0204 bGHRH plasmid (SEQ ID#19).

69. The method of claim 66, wherein the isolated nucleic acid expression construct is a TI-GHRH plasmid (SEQ ID#12), TV-GHRH Plasmid (SEQ ID#13), 15/27/28 GHRH plasmid (SEQ ID#14), pSP-wt-GHRH plasmid, pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

70. The method of claim 66, wherein the isolated nucleic acid expression construct further comprises, poly-L-glutamate.

71. The method of claim 66, wherein the farm animals comprise bovine.

72. A method of increasing milk production in a dairy cow comprising: delivering into tissues of the dairy cow an isolated nucleic acid expression construct that encodes a growth-hormone-releasing-hormone (“GHRH”) or functional biological equivalent thereof;
wherein:
delivering is via an in vivo electroporation method;
the isolated nucleic acid expression construct is delivered in a single dose; and
the encoded GHRH or functional biological equivalent thereof is of formula (SEQ ID No: 6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

73. The method of claim 72, wherein the single dose comprises about a 2 mg quantity of nucleic acid expression construct.

74. The method of claim 72, wherein the isolated nucleic acid expression construct is a HV-GHRH plasmid (SEQ ID#11), or an optimized pAV0204 bGHRH plasmid (SEQ ID#19).

75. The method of claim 72, wherein the isolated nucleic acid expression construct is a TI-GHRH plasmid (SEQ ID#12), TV-GHRH Plasmid (SEQ ID#13), 15/27/28 GHRH plasmid (SEQ ID#14), pSP-wt-GHRH plasmid, pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

76. The method of claim 72, wherein the isolated nucleic acid expression construct further comprises, poly-L-glutamate.

77. A method of decreasing an involuntary cull in farm animals comprising: delivering into the farm animals a growth hormone secretagogue molecule or functional biological equivalent thereof; wherein the involuntary cull comprises infection, disease, morbidity, or mortality of the farm animals; and the growth hormone secretagogue molecule or functional biological equivalent thereof facilitates growth hormone (“GH”) secretion in the farm animal.

78. The method of claim 77, wherein delivering into the tissue of the farm animals the growth hormone secretagogue molecule is via an electroporation method, a viral vector or nucleic acid expression construct, in conjunction with a carrier, by parenteral route, orally, or a combination thereof.

79. The method of claim 77, wherein the involuntary cull comprises mortality at birth of newborns of the farm animals.

80. The method of claim 77, wherein the involuntary cull comprises post-natal mortality of newborns of the farm animals.

81. The method of claim 77, wherein the growth hormone secretagogue molecule comprises a growth hormone releasing hormone (“GHRH”) or functional biological equivalent thereof.

82. The method of claim 77, wherein growth hormone secretagogue comprises an isolated nucleic acid expression construct that encodes the growth hormone releasing hormone (“GHRH”) or functional biological equivalent thereof.

83. The method of claim 81, wherein the isolated nucleic acid expression construct comprises a HV-GHRH plasmid (SEQ ID#11).

84. The method of claim 81, wherein the isolated nucleic acid expression construct is an optimized pAV0204 bGHRH plasmid (SEQ ID#19), TI-GHRH plasmid (SEQ ID#12), TV-GHRH Plasmid (SEQ ID#13), 15/27/28 GHRH plasmid (SEQ ID#14), pSP-wt-GHRH plasmid, an optimized pAV0202 mGHRH plasmid (SEQ ID#17), pAV0203 rGHRH plasmid (SEQ ID#18), pAV0205 oGHRH plasmid (SEQID#20), pAV0206 cGHRH plasmid (SEQ ID#21), or pAV0207 pGHRH plasmid (SEQ ID#28).

85. The method of claim 81, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

86. The method of claim 81, wherein the encoded GHRH or functional biological equivalent thereof is of formula (SEQ ID No: 6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

87. The method of claim 77, wherein the farm animals comprises ruminant animals, food animals, or work animals.

88. The method of claim 77, wherein the farm animals comprise dairy cows.

89. A method of improving a body condition score (“BCS”) in farm animals comprising: delivering into the farm animals a growth hormone secretagogue molecule or functional biological equivalent thereof; wherein the BSC is an aid used to evaluate an overall nutritional state of the farm animals, and the growth hormone secretagogue molecule or functional biological equivalent thereof facilitates growth hormone (“GH”) secretion in the farm animal.

90. The method of claim 89, wherein delivering into the tissue of the farm animals the isolated nucleic acid expression construct is via electroporation method, a viral vector, in conjunction with a carrier, by parenteral route, orally, or a combination thereof.

91. The method of claim 89, wherein the growth hormone secretagogue molecule comprises a growth hormone releasing hormone (“GHRH”) or functional biological equivalent thereof.

92. The method of claim 89, wherein growth hormone secretagogue comprises an isolated nucleic acid expression construct that encodes the growth hormone releasing hormone (“GHRH”) or functional biological equivalent thereof.

93. The method of claim 89, wherein growth hormone secretagogue comprises an isolated nucleic acid expression construct that encodes the growth hormone releasing hormone (“GHRH”) or functional biological equivalent thereof.

94. The method of claim 93, wherein the isolated nucleic acid expression construct comprises a HV-GHRH plasmid (SEQ ID#11).

95. The method of claim 93, wherein the isolated nucleic acid expression construct is an optimized pAV0204 bGHRH plasmid (SEQ ID#19), TI-GHRH plasmid (SEQ ID#12), TV-GHRH Plasmid (SEQ ID#13), 15/27/28 GHRH plasmid (SEQ ID#14), pSP-wt-GHRH plasmid, an optimized pAV0202 mGHRH plasmid (SEQ ID#17), pAV0203 rGHRH plasmid (SEQ ID#18), pAV0205 oGHRH plasmid (SEQ ID#20), pAV0206 cGHRH plasmid (SEQ ID#21), or pAV0207 pGHRH plasmid (SEQ ID#28).

96. The method of claim 93, wherein the encoded GHRH is a biologically active polypeptide; and the encoded functional biological equivalent of GHRH is a polypeptide that has been engineered to contain a distinct amino acid sequence while simultaneously having similar or improved biologically activity when compared to the GHRH polypeptide.

97. The method of claim 93, wherein the encoded GHRH or functional biological equivalent thereof is of formula (SEQ ID No: 6):
—X₋₁—X₂-DAIFTNSYRKVL-X₃-QLSARKLLQDI-X₄—X₅-RQQGERNQEQGA-OH
wherein the formula has the following characteristics:
X₁ is a D-or L-isomer of the amino acid tyrosine (“Y”), or histidine (“H”);
X₂ is a D-or L-isomer of the amino acid alanine (“A”), valine (“V”), or isoleucine (“I”);
X₃ is a D-or L-isomer of the amino acid alanine (“A”) or glycine (“G”);
X₄ is a D-or L-isomer of the amino acid methionine (“M”), or leucine (“L”);
X₅ is a D-or L-isomer of the amino acid serine (“S”) or asparagine (“N”);
or a combination thereof.

98. The method of claim 89, wherein the farm animals comprises ruminant animals, food animals, or work animals.

99. The method of claim 89, wherein the farm animals comprise dairy cows.
